(19) United States
(12) Patent Application Publication
(10) Pub. No.: US 2014/0037661 A1
(43) Pub. Date: Feb. 6, 2014

(54) Wheat Antigens and Peptides for Diagnosis of Wheat Induced Hypersensitivity

(71) Applicants: Alexandra Baar, Gerasdorf (AT); Rudolf Valenta, Theresienfeld (AT); Sandra Pahr, Kirchschlag (AT); Bharani Srinivasan, Wien (AT); Susanne Vrtala, Wien (AT)

(72) Inventors: Alexandra Baar, Gerasdorf (AT); Rudolf Valenta, Theresienfeld (AT); Sandra Pahr, Kirchschlag (AT); Bharani Srinivasan, Wien (AT); Susanne Vrtala, Wien (AT)

(73) Assignee: PHADIA AB, Uppsala (SE)

(21) Appl. No.: 13/984,207

(22) PCT Filed: Feb. 8, 2012
(86) PCT No.: PCT/SE2012/050121

§ 371 Date: Oct. 25, 2013

(30) Foreign Application Priority Data
Feb. 8, 2011 (SE) 1150096-4

(51) Int. Cl. C07K 14/415; G01N 33/50

(52) U.S. Cl. CPC C07K 14/415; G01N 33/5008
USPC 424/185.1; 530/375; 536/23.6; 514/21.2; 435/29; 435/7.9; 435/23

(57) ABSTRACT

The present invention relates to the field of different wheat hypersensitivities, particularly with antigens and peptides for discrimination of different forms of these diseases. The invention relates to the identification of novel wheat allergens and the use thereof in therapy and diagnosis of celiac disease, dermatitis herpetiformis, and IgE-mediated allergy. Furthermore, the present invention provides the use of known peptides and proteins in therapy and diagnosis. The invention also relates to methods for diagnosis and treatment of celiac disease, dermatitis herpetiformis, and IgE-mediated allergy.

### FIELD OF THE INVENTION

The present invention relates to the field of different forms of wheat hypersensitivities, particularly with antigens and peptides for discrimination of different forms of these diseases.

### BACKGROUND

According to Gell and Coombs classification (which is a classification of immune mechanisms of tissue injury), four types of hypersensitivity exist: type I, immediate hypersensitivity reactions, mediated by interaction of IgE antibody and antigen and release of histamine and other mediators; type II, antibody-mediated hypersensitivity reactions, due to antibody-antigen interactions on cell surfaces; type III, immune complex, local or general inflammatory responses due to formation of circulating immune complexes and their deposition in tissues; and type IV cell-mediated hypersensitivity reactions, initiated by sensitized T lymphocytes either by release of lymphokines or by T-cell-mediated cytotoxicity.

Wheat (Triticum aestivum) can cause different forms of hypersensitivities. It can cause three distinct IgE-mediated allergies, wheat pollen allergy, Baker's Asthma and wheat food allergy. The wheat pollen allergy belongs to the group of grass pollen allergies. Baker's asthma is a respiratory allergy which is caused by wheat flour; it is an important occupational disease that often affects bakers, millers or confectioners. Wheat induced food allergy is very common and occurs after ingestion of wheat containing food, leading to diverse clinical manifestations including eczema, urticaria, gastrointestinal symptoms, conjunctivitis and many other symptoms (1). Additional to the IgE mediated wheat allergy there exists a hypersensitivity to wheat, the celiac disease, which is characterized by IgA antibodies and T-cell reactivity against wheat proteins and development of auto reactive IgA antibodies against several intestinal proteins (2, 3). It is an inflammatory hypersensitivity to wheat which causes villous atrophy in the small intestine and leads to symptoms like chronic diarrhoea or constipation, malnutrition, anaemia, fatigue, growth retardation and migraine (4).

Since wheat (Triticum aestivum) and wheat products are a major element in nutrition, avoidance of wheat products is currently the only therapy for patients suffering from wheat induced hypersensitivities. Antigen specific approaches would require a detailed knowledge and availability of the hypersensitivity causing protein. To date there is a lack of defined proteins and peptides to be used as diagnostic tool to discriminate between the different forms of hypersensitivities to wheat. Therefore, precise diagnosis still relies on specific inhalation challenge in case of respiratory allergy to wheat flour, double-blind placebo-controlled food challenge (DBPCFC) in case of suspected food allergy, and diet followed by rechallenge and/or intestinal biopsy for celiac disease. Constantin et al (5) identified specific recombinant wheat flour allergens which are recognized by Baker's asthma patients, but not by wheat food allergic patients. They showed the usefulness of micro-arrayed recombinant allergens in contrast to natural extracts. However, the panel of allergens was incomplete and there is therefore a need to identify more antigens and peptides that are involved in wheat food allergy or celiac disease and to establish methods and diagnostic tests to differentiate patients suffering from the different forms of wheat hypersensitivities. In addition, there is a need to use such wheat antigens and peptides for treatment of wheat mediated hypersensitivities.

### SUMMARY OF THE INVENTION

The object of the present invention is to overcome the problems related to the prior art as described above. The present invention provides polypeptides and nucleic acid sequences, which are related to different forms of wheat hypersensitivities, and which may be used for therapy and diagnosis of different forms of wheat hypersensitivities.

According to one aspect of the present invention, an isolated polypeptide is provided that comprises the amino acid sequence according to any one of SEQ ID NO: 26-50, 62-86, and 89-110.

In one embodiment, the polypeptide is characterised in that it is isolated from wheat or recombinantly produced. Alternatively, the polypeptide may be produced by chemical synthesis.

A further embodiment of the present invention provides an isolated nucleic acid molecule encoding the polypeptide as described above. For example, the nucleic acid may have the nucleotide sequence according to any one of SEQ ID NO: 1-25.

According to another aspect of the present invention, the polypeptide as described above, or a fragment or variant thereof sharing epitopes for antibodies with said polypeptide, is for use in therapy or diagnosis.

More particulary, the polypeptide or a fragment or variant thereof sharing epitopes for antibodies with said polypeptide, is for use in therapy or diagnosis of celiac disease, dermatitis herpetiformis, or IgE-mediated allergy. Dermatitis herpetiformis is a skin disease, which is associated with celiac disease.

The present invention further provides an isolated polypeptide comprising the amino acid sequence according to any one of SEQ ID NO: 51-61, 87 and 88, or a fragment or variant thereof sharing epitopes for antibodies with said polypeptide, for use in therapy or diagnosis.

More specifically, such an isolated polypeptide comprising the amino acid sequence according to any one of SEQ ID NO: 51-61, 87 and 88 or a fragment or variant thereof sharing epitopes for antibodies with said polypeptide, is for use in therapy or diagnosis of celiac disease, dermatitis herpetiformis, or IgE-mediated allergy. Further, according to one embodiment, the use in therapy comprises tolerance induction or prophylactic treatment.

According to yet another aspect, the present invention provides a pharmaceutical composition comprising a polypeptide having the amino acid sequence according to any one of SEQ ID NO: 26-110, or a hypoallergenic form of said polypeptide that is modified to abrogate or attenuate its T cell-, IgA- or IgE-binding response, and optionally pharmaceutically acceptable excipients, carriers, buffers and/or diluents.

In one embodiment, the hypoallergenic form of the polypeptide comprised by the pharmaceutical composition is modified by fragmentation, truncation or tandemerization of the molecule, deletion of internal segments, domain rearrangment, substitution of amino acid residues, disruption of disulfide bridges.

A further aspect of the present invention provides a method for producing an allergen composition comprising the step of adding a polypeptide having the amino acid sequence according to any one of SEQ ID NO: 26-110, or a fragment or variant thereof sharing epitopes for antibodies with said polypeptide, to a composition comprising an allergen extract and/or at least one purified allergen component.

Further provided is an allergen composition obtainable with the above-described method.

The present invention also provides a method for in vitro diagnosis of celiac disease comprising
contacting a body fluid or tissue sample from a mammal suspected of having celiac disease with at least one polypeptide having the amino acid sequence according to any one of SEQ ID NO: 62-110 or a fragment or variant thereof sharing epitopes for antibodies with said polypeptide; and
measuring activated T cells in the sample, such as by use of a lymphocyte proliferation assay, a FACS analysis of the cell activation, or by measuring cytokine release;
   wherein the presence of activated T cells is indicative of celiac disease.

T-cell number and function may for example be monitored by assays that detect T cells by an activity such as cytokine production, proliferation, or cytotoxicity (9, 10).

It has previously been described that certain T cells from celiac mucosa produce cytokines with Th1 or Th0 profile, particularly interferon-gamma (IFN-γ). This cytokine, particularly in combination with TNF-alpha, might be involved in several pathological features of the celiac lesion (10).

In a similar scenario, it has been shown that IFN-γ derived from T cells facilitates allergen penetration through respiratory epithelium cells and thereby augment allergic inflammation (11). Further, it has been shown that IFN-γ-containing culture supernatants from peripheral blood mononuclear cells stimulated by a certain autoantigen caused disintegration of respiratory epithelial cell layers and apoptosis of skin keratinocytes. This damage could be inhibited with a neutralizing anti-IFN-γ antibody (12).

Consequently, the present invention also provides a method for in vitro diagnosis of celiac disease comprising
contacting leukocytes from a mammal suspected of having celiac disease with at least one polypeptide having the amino acid sequence according to any one of SEQ ID NO: 62-110 or a fragment or variant thereof sharing epitopes for antibodies with said polypeptide, in a medium;
contacting a cell sample from said mammal with the medium; and
measuring the presence of interferon-gamma or other cell-damaging substance(s) in the cell sample;
   wherein the presence of interferon-gamma or other cell-damaging substance(s) is indicative of celiac disease.

In an embodiment, the leukocytes producing cell-damaging substances are lymphocytes, such as different types of T cells.

In an embodiment, the medium, in which leukocytes are brought into contact with the polypeptide(s), is a body fluid or tissue sample, and before bringing the cell sample into contact with the body fluid or tissue sample, a supernatant is prepared from the body fluid or tissue sample, and the cell sample is contacted with the supernatant. The presence of interferon-gamma or other cell-damaging substance(s) in the cell sample is then measured.

In a preferred embodiment of this method, the cell sample comprises intestinal epithelial cells.

The cell damage resulting from the effect of the cell-damaging substance(s) may include disintegration of cell layers and apoptosis.

The invention further provides a method for in vitro diagnosis of celiac disease, dermatitis herpetiformis, or IgE-mediated allergy, comprising
contacting a body fluid or tissue sample from a mammal suspected of having celiac disease or IgE-mediated allergy with at least one polypeptide having the amino acid sequence according to any one of SEQ ID NO: 26-110 or a fragment or variant thereof sharing epitopes for antibodies with said polypeptide; and
detecting the presence, in the sample, of IgA or IgE antibodies specifically binding to said polypeptide or polypeptides;
   wherein the presence of such antibodies specifically binding to said polypeptide or polypeptides is indicative of celiac disease, dermatitis herpetiformis, or IgE-mediated allergy.

According to a preferred embodiment of the invention, the IgE-mediated allergy is wheat food allergy.

According to a further aspect, a diagnostic kit is provided for performing the methods of the invention, comprising a polypeptide having the amino acid sequence according to any one of SEQ ID NO: 26-110 or a fragment or variant thereof sharing epitopes for antibodies with said polypeptide, or a pharmaceutical composition as described above.

### Definitions

All words and terms used in the present specification are intended to have the meaning usually given to them in the relevant art. However, for the sake of clarity, a few terms are specifically clarified below.

The expression “a fragment or variant of a polypeptide sharing epitopes for antibodies with said polypeptide” has the meaning as defined in WO2008/079095.

### BRIEF DESCRIPTION OF THE DRAWINGS

FIG. 1. cDNA and deduced amino acid sequences of LMW Glutenin GluB3-23 and C175. The C-terminal part shown in bold letters is the clone 175 sequence.

FIG. 2. Nucleotide sequence of Glu-B1 aligned with amino acid sequence of the IgE-reactive clone 43.

FIG. 3. Nucleotide sequence of Glu-B1 aligned with amino acid sequence of the IgE-reactive clone 82.

FIG. 4. The nucleotide sequence and deduced amino acid sequence of the clone 84-derived allergen.

FIG. 5. Deduced amino acid sequences of IgE reactive cDNA clones coding for wheat allergens.

FIG. 6. Amino acid sequences of IgE reactive wheat epitopes.

FIG. 7a. Domain structure of the natural GluB3-23, the recombinant GluB3-23 and C175.

FIGS. 7b and 7c: Mass spectrometry (MS) of the purified C175 and GluB3-23. The mass/charge ratio is shown on the x-axis and the intensity is displayed on the y-axis and is shown in arbitrary units.

FIG. 8a. Domain structure of the natural Glu-B1 and the recombinant proteins

FIG. 8b. Mass spectrometry (MS) of the purified mal 43 (clone 43). The mass/charge ratio is shown on the x-axis and the intensity is displayed on the y-axis and is shown in arbitrary units.

FIG. 8c. Mass spectrometry (MS) of the purified mal 82 (clone 82). The mass/charge ratio is shown on the x-axis and the intensity is displayed on the y-axis and is shown in arbitrary units.

FIG. 9a. The C-terminal acidic extension domain and a part of the thionin domain are identified as IgE epitope-containing portion.

FIG. 9b. Mass spectrometry of the recombinant allergen α-purothionin. The mass/charge ratio is shown on the x-axis and the intensity is displayed on the y-axis as a percentage of the most intensive signal obtained in the investigated mass range.

FIG. 10. IgE reactivity of patients suffering from wheat food allergy. Dot blotted purified recombinant proteins (GluB3-23 and C175), aqueous wheat seed (WSE) extract and human serum albumin (HSA) were incubated with sera from patients suffering from wheat food allergy. Bound IgE Abs were detected with ¹²⁵I labeled anti human IgE Abs and visualized by autoradiography.

FIG. 11. IgE reactivity of patients suffering from wheat food allergy. Dot blotted purified recombinant proteins (GluB3-23, C175, mal 43 and mal 82), aqueous wheat seed (WSE) extract and human serum albumin (HSA) were incubated with sera from patients suffering from wheat food allergy. Bound IgE Abs were detected with ¹²⁵I labelled anti human IgE Abs and visualized by autoradiography. Frequencies of recognition are displayed in the right margin.

FIG. 12. IgE reactivity of patients suffering from wheat food allergy. Wheat seed extract, HSA, purified alpha purothionin were dotted onto nitrocellulose membrane strips and incubated with sera from wheat food allergic patients. Bound IgE antibodies were detected with ¹²⁵I-labelled anti-human IgE antibodies and visualized by autoradiography.

FIG. 13. Sequence alignment of GluB3-23 with related proteins in rye, barley, oat, spelt and rice. A point indicates identity and a dash displays a gap. At the end of the alignment the identity to GluB2-23 is shown in percentage.

FIG. 14. Multiple sequence alignment of the clone 84-derived allergen alpha purothionin with homologous proteins in other plants. The amino acid sequence (single letter code) of wheat alpha purothionin was aligned with purothionins in wheat (gi|4007850), rye (gi|4007745), barley (gi|246215), oat (gi|21069045), goatgrass (gi|1052551), rice (gi|215768993), sage (gi|77543393), thale cress (gi|21553588), mustard (gi|120564556), pieplant (gi|197312881). Frequencies of recognition are displayed in the right margin.

FIG. 15. IgA reactivity of celiac disease patients to purified wheat proteins. ELISA measurements of IgA reactivity of celiac disease patients' sera and and a control patient's serum to wheat proteins coated onto ELISA plates. After incubation with patients' sera, the bound IgA was detected using mouse anti-human IgA1/A2 as primary antibody and HRP conjugated sheep anti-mouse IgG as detection antibody. The colour reaction was measured at 405 nm. Wheat and control proteins are indicated on the X-axis and the legend on the right-hand corner indicates patients. Abbreviations used: HSA—Human serum Albumin, GG1 -Gamma gliadin 1, GG2—Gamma gliadin 2, P—Patients, CD—celiac disease positive, GFD—gluten free diet.

FIG. 16. IgA reactivity of celiac disease patients to synthesized gamma gliadin 1 peptides.

FIG. 17. IgG reactivity of celiac disease patients to synthesized gamma gliadin 1 peptides.

FIG. 18. IgA reactivity of Dermatitis herpetiformis patient to recombinant gamma gliadins.

### DETAILED DESCRIPTION OF THE INVENTION

To date, there is only a limited set of antigens and peptides to be used as diagnostic tool to discriminate between the different forms of wheat induced hypersensitivities. This led the present inventors to look for novel and well-defined wheat antigens and peptides that can be used for the diagnosis of various wheat hypersensitivities, by screening of a wheat cDNA library and use of classical immunochemical approaches and ion exchange chromatography generated gluten fractions, with well characterized patients' sera. The identification of wheat antigens and peptides, the production and characterization of recombinant proteins permits creating tools for diagnosis (development of chips) and for treatment of such wheat induced hypersensitivities.

The examples below illustrate the present invention with the isolation and use of the nucleic acid sequences and polypeptides of the invention. The examples are only illustrative and should not be considered as limiting the invention, which is defined by the scope of the appended claims.

### Example 1

### Construction and Screening of a λgt11 cDNA Library From Wheat Seeds

In order to find new wheat allergens, total RNA from wheat seeds were extracted and a λgt11 cDNA library was constructed as described previously (5). E. coli Y1090 were infected with 7×10⁵ PFU of recombinant phages and immunoscreened with serum IgE of three patients suffering from wheat food allergy. After pre-adsorption with nitrocellulose filters, containing λgt11 phages, the 1:10 serum dilution was added to the filters prepared from the already titrated phage clones. Bound IgE antibodies were detected with 1:10 diluted ¹²⁵I-labeled α-human IgE and visualized by autoradiography. The IgE-reactive phage clones were selected for further re-cloning and their DNA was PCR-amplified using Platinum PCR SuperMix (Invitrogen) with λgt11 primers and sequenced (VBC-Biotech). The obtained sequences were compared with sequences submitted to the GenBank database at the National Center for Biotechnology Information (NCBI) to find homologous proteins. In some cases we obtained only IgE reactive epitopes without identifying a corresponding protein. The list of all IgE reactive clones is shown in Table 1.

### Example 2

### Expression and Purification

### Clone 175 and Glub3-23

The clone 175 sequence (SEQ ID NO: 1) containing 537 nucleotides and the corresponding full sequence GluB3-23 (SEQ ID NO: 51) involving 1107 nucleotides and six His codons were cloned into pET17b E. coli expression vectors. The pET 17b-C175 and the pET 17b-GluB3-23 construct were transformed into E. coli BL21 (DE3). The transformed cells were grown in 1 liter Luria Broth medium containing 100 mg/l ampicillin at 37° C. The cells were grown until an OD₆₀₀ of 0.4-0.6, and then the over expression was induced by addition of isopropyl β-D-thiogalactopyranoside (IPTG) to a final concentration of 0.5 mM. Afterwards the bacteria were grown for 4 additional hours; cells were harvested by centrifugation and frozen over night at −20° C. A cleared cell lysate was prepared and a NiNTA-chromatography was performed according to QIAexpressionist handbook (QIAGEN, Hilden, Germany). The protein containing fractions were pooled and dialysed against 10 mM NaH₂PO₄. The protein concentration was determined with a BCA Assay Kit (Novagen).

### Clones 43 and 82

The clone 43 sequence (SEQ ID NO: 2) containing 828 nucleotides and the clone 82 sequence (SEQ ID NO: 3) involving 588 nucleotides plus six His codons were cloned into pMal-c4x E. coli expression vectors (GeneScript USA Inc.). The pMAL-c4x-43 and pMAL-c4x-82 constructs were transformed into E. coli BL21 (DE3) and grown in 1 liter Luria Broth+glucose medium containing 100 mg/l ampicillin at 37° C. The cells were grown until an OD₆₀₀ of 0.4-0.6, and then the over expression was induced by addition of isopropyl β-D-thiogalactopyranoside (IPTG) to a final concentration of 0.5 mM. Afterwards the bacteria were grown for 4 additional hours; cells were harvested by centrifugation and frozen over night at −20° C. A cleared cell lysate was prepared and a NiNTA-chromatography was performed according to QIAexpressionist handbook (QIAGEN, Hilden, Germany). The protein containing fractions were pooled and dialysed against 10 mM NaH₂PO₄. The protein concentration was determined with a BCA Assay Kit (Novagen).

### α-Purothionin

The clone 84-derived allergen was expressed as a recombinant protein with a C-terminal hexahistidine tag in E. coli BL21 (DE3) cells. The pET 17b-α-purothionin construct was transformed into Bl21 (DE3) cells. The transformed E. coli cells were grown in 250 ml LB medium containing 250 μl (100 mg/ml) ampicillin at 37° C. to an optical density (600 nm) of 0.6 and protein expression was induced by addition of 125 μl (1M) isopropyl-beta-D-thiogalactosidase (IPTG). E. coli cells were harvested after 4 hours by centrifugation at 3500 rpm for 15 min at 4° C. The protein was purified by nickel affinity chromatography from the soluble fraction (Quiagen, Hilden, Germany). The allergen was dissolved and stored in 10 mM NaH₂PO4 buffer pH 4.0 at −20° C. The concentrations of the purified allergens were determined by BCA assay (Pierce, Rockford, Ill.).

### Example 3

### Characterization of the Recombinant Proteins

### C175 and GluB3-23

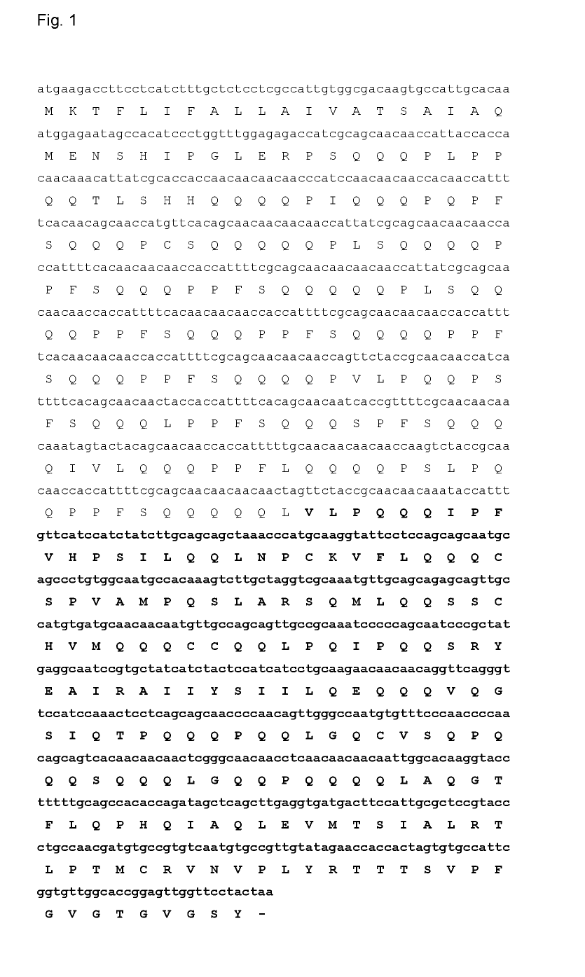

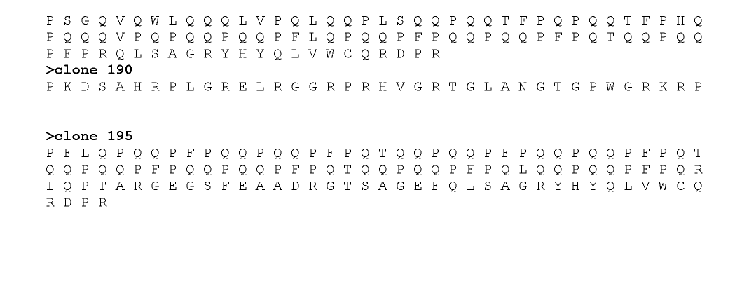

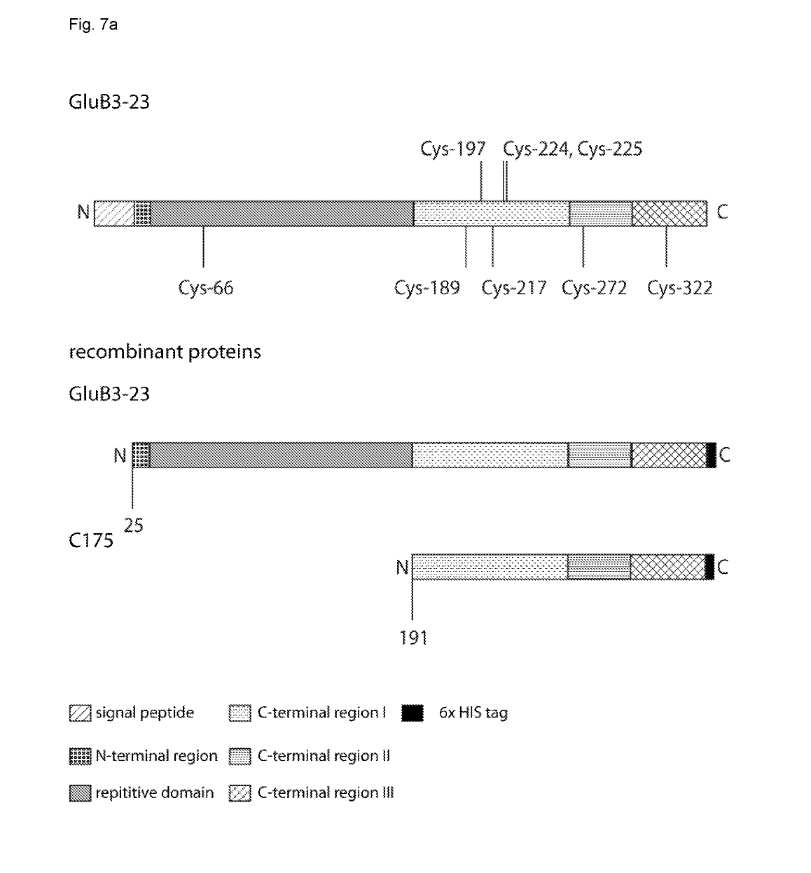

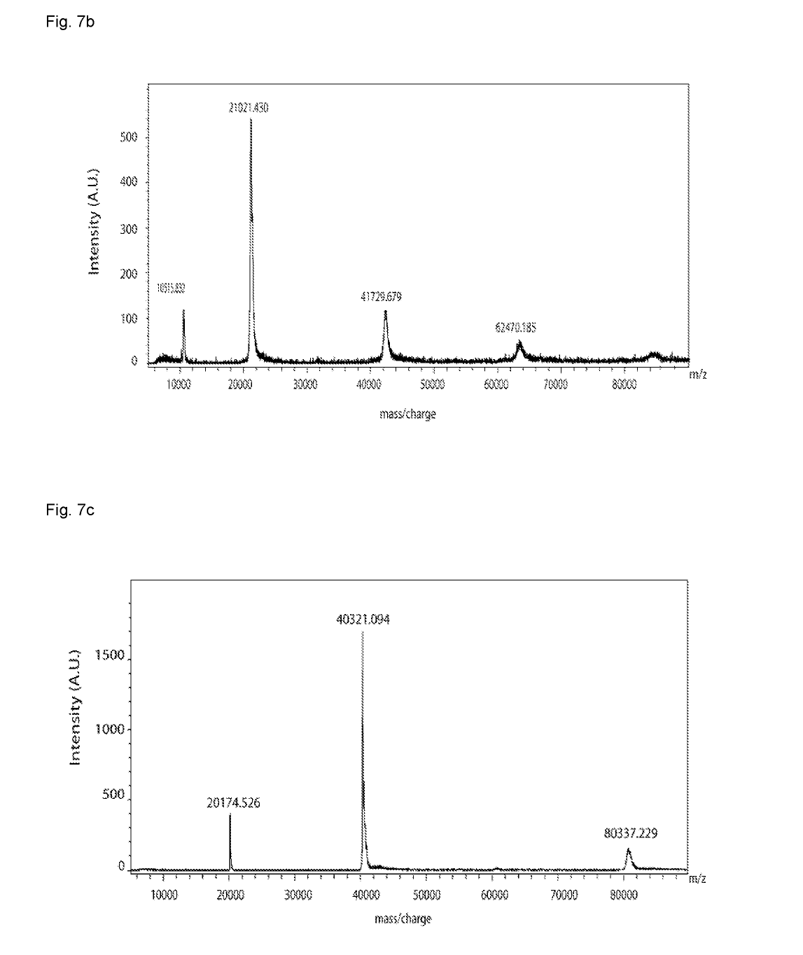

Sequence analysis showed that GluB3-23 is an s-LMW glutenin that contains eight cysteine residues for building intramolecular disulphide bonds for stability and intermolecular disulphide bonds with other LMW and BMW glutenin subunits to form macropolymers. The natural protein consists of a signal peptide, an N-terminal region, a repetitive domain and three C-terminal regions. The recombinant C175 protein is comprised by the three C-terminal regions and the hexa-histidine tag (FIG. 1). The recombinant Glub3-23 contains all regions of the natural Glub3-23 without the signal peptide and plus a hexa-histidine-tag (FIG. 7a).

For the recombinant C175 protein a molecular weight of 20.8 kDa and a theoretical pI of 8.81 was calculated and for the recombinant GluB3-23 a molecular weight was assessed at 40.33 kDa and the theoretical pI at 8.73. The purity and molecular mass was controlled by SDS-PAGE and Coomassie Brilliant Blue staining (Fling, Bradford). C175 provided a clear band at approximately 21 kDa and GluB3-23 at 40 kDa. To achieve information about the polymerization behavior of the proteins SDS PAGE silver staining was performed according to BIO RAD silver stain Plus Handbook under reducing and non reducing conditions. For reducing conditions, a sample buffer containing β-Mercaptoethanol was used and samples were boiled at 95° C. for 5 minutes; for non reducing conditions, a sample buffer without (3-Mercaptoethanol was used. Under non reducing conditions C175 provided bands at approximately 20 kDa, 40 kDa and 250 kDa, which indicates that C175 forms di-, tri- and polymers by disulfide bonds. Glub3-23 also forms polymers, shown by bands at approximately 40kDa and 250 kDa under non reducing conditions.

Mass spectrometry was performed as described previously (6). In FIG. 7b and FIG. 7c mass spectrometry (MS) of the purified C175 and GluB3-23 is shown. The peak with the highest intensity indicates the protein size. C175 shows the peak at 21021.430 Da and GluB3-23 at 40321.094 Da, which correlates with the calculated mass.

### Mal43 and Mal82

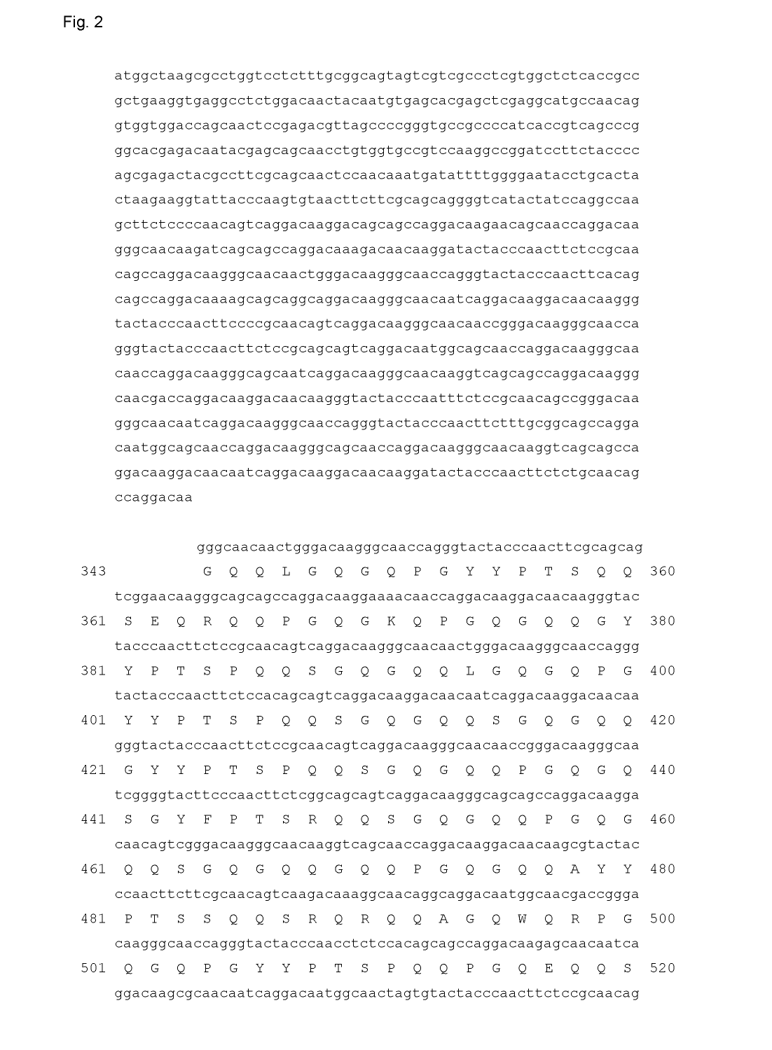

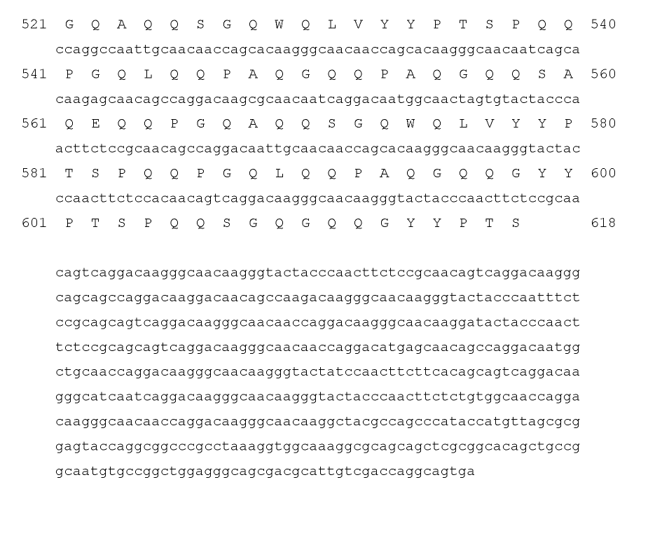

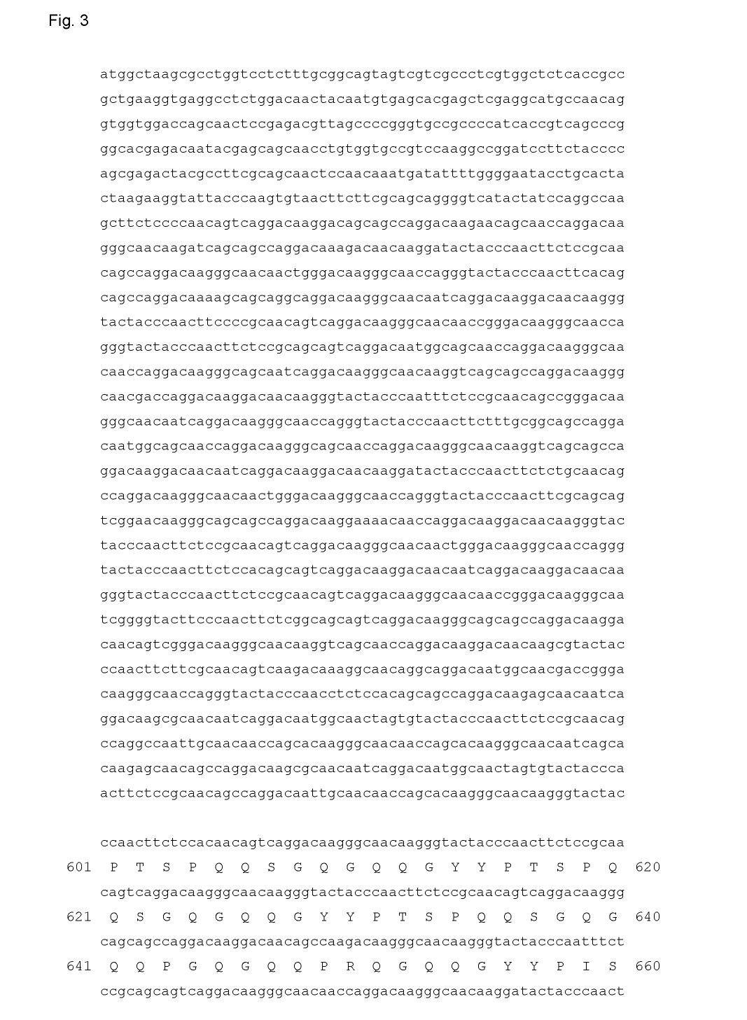

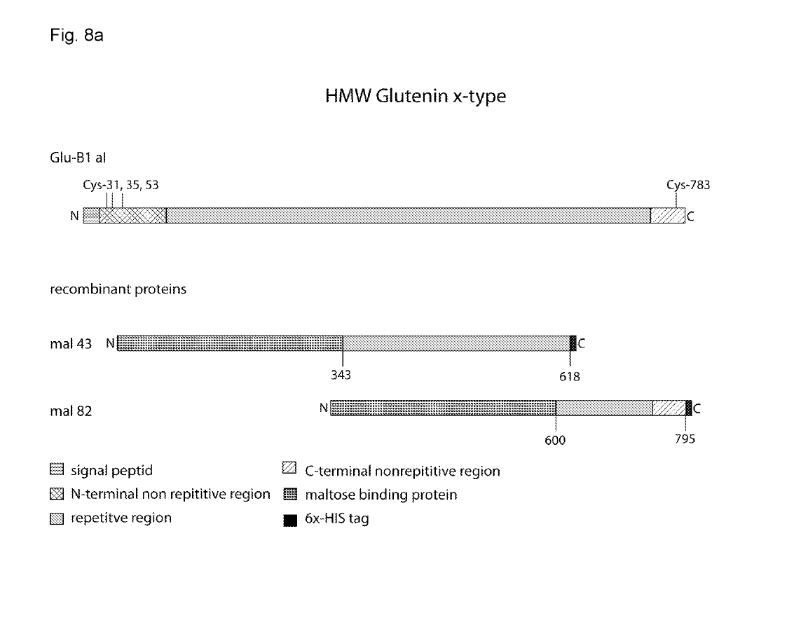

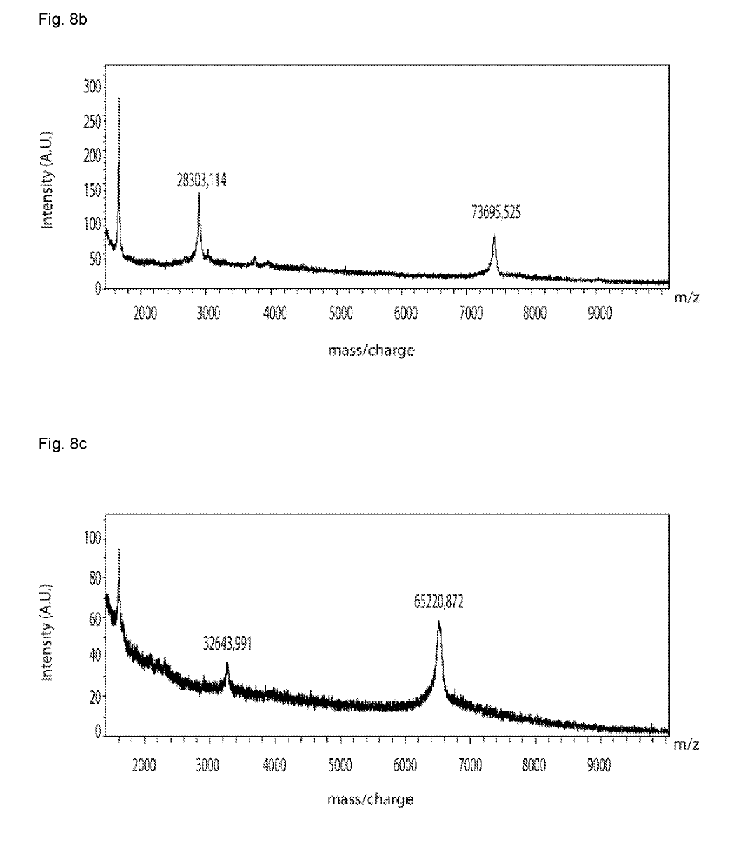

The recombinant proteins corresponding to clones 43 and 82 featured an N-terminal maltose binding protein tag (MBP-tag) shown in FIG. 8a, resulting from the pMAL-c4x vector. For mal43 a molecular weight of 73.3 kDa and a theoretical pI of 5.81 and for mal82 a molecular weight of 64.6 kDa and a theoretical pI of 5.99 were calculated. The sequence analysis of the full length BMW Glu-B1 (corresponding to SEQ ID No: 52) showed, that the protein is an x-type HMW protein with four cysteine residues for disulphide bond forming. The natural Glu-B1 contains a signal peptide, an N-terminal non repetitive domain, a large repetitive domain and a C-terminal non repetitive domain. The recombinant mal43 consists of the MBP, a part of the repetitive domain and the hexa-histidine tag. The recombinant mal82 is made up of the MBP, a part of the repetitive region, the C-terminal non repetitive region and the hexa-histidine tag (FIG. 8a). In FIG. 2, the nucleotide sequence of Glu-B1 is aligned with the deduced amino acid sequence of clone 43, and in FIG. 3, the nucleotide sequence of Glu-B1 is aligned with the deduced amino acid sequence of clone 82.

The purity and molecular mass was controlled by SDS-PAGE and Coomassie Brilliant Blue staining (Fling, Bradford). Mal43 provided a clear band at approximately 73 kDa and mal82 at 64 kDa. Mass spectrometry was performed as described previously (6). In FIG. 8b and FIG. 8c mass spectrometry (MS) of the purified mal43 and mal82 is shown. The peak at 73695.525 Da displays the size of mal43. In FIG. 8c the peak at 65220.872 shows the molecular weight of mal82. These results correlate with the calculated molecular weights.

### α-Purothionin

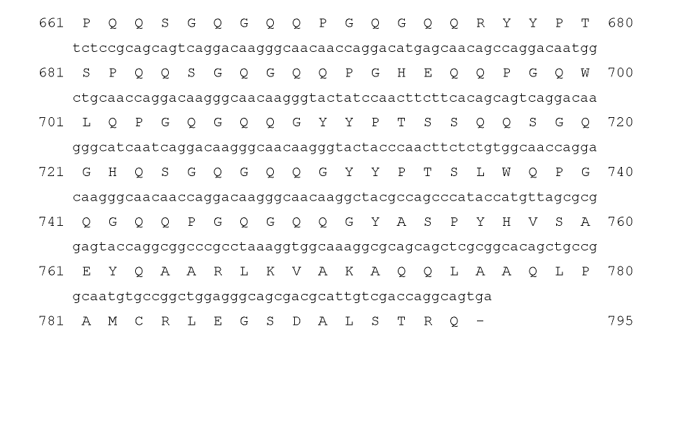

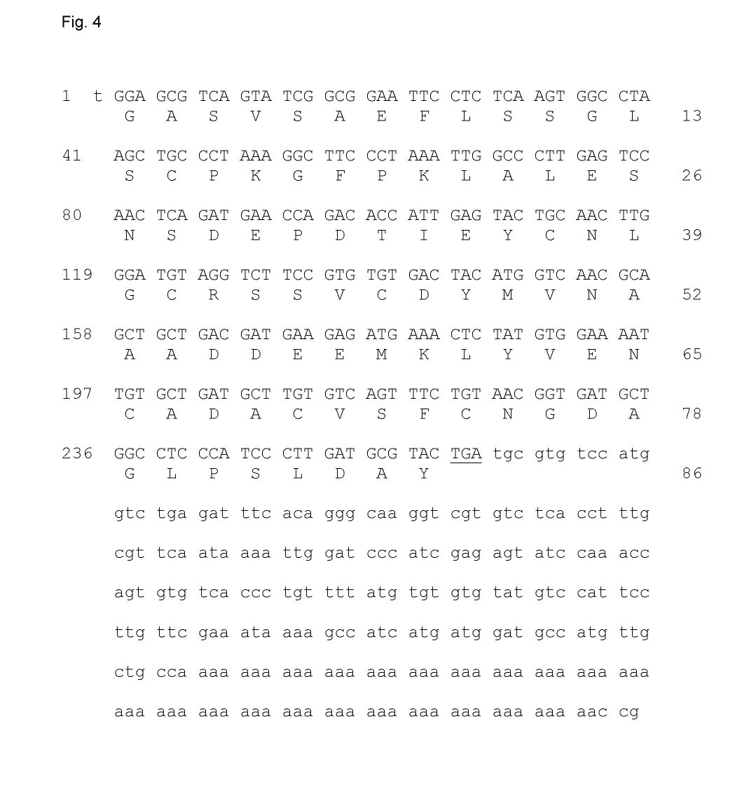

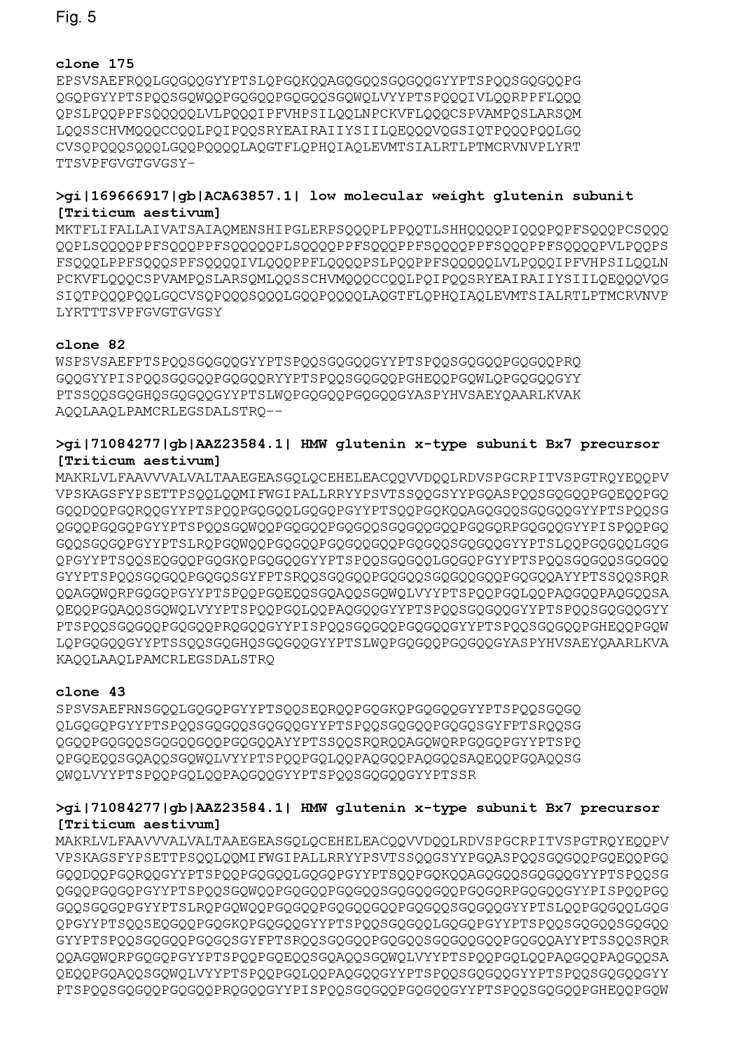

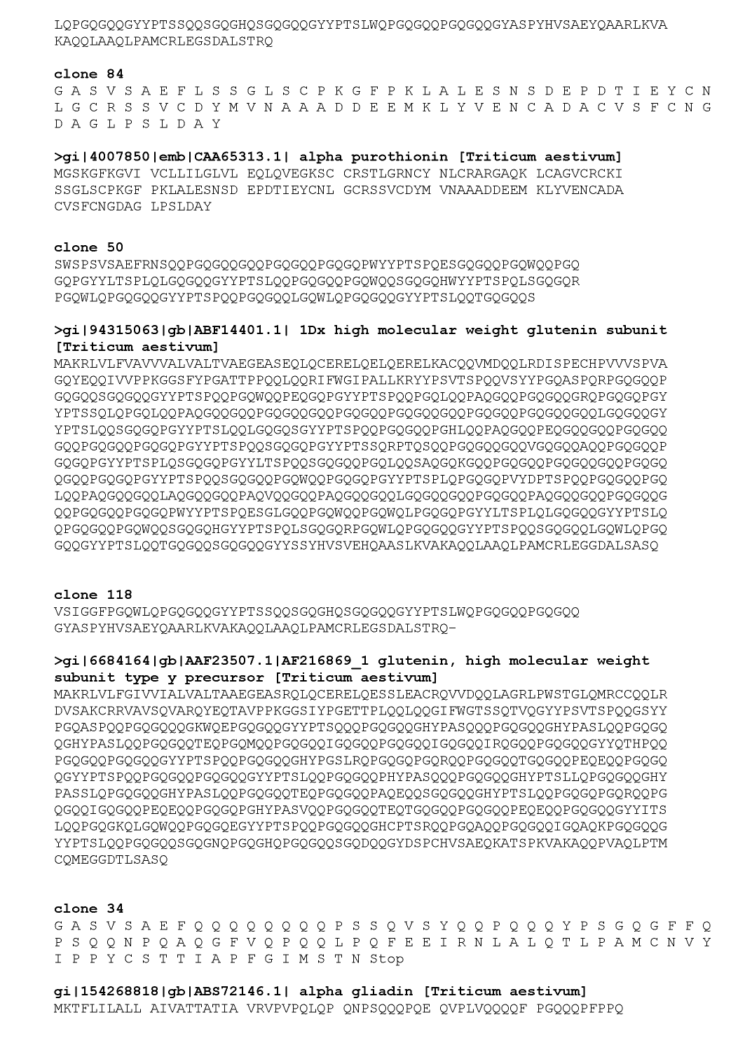

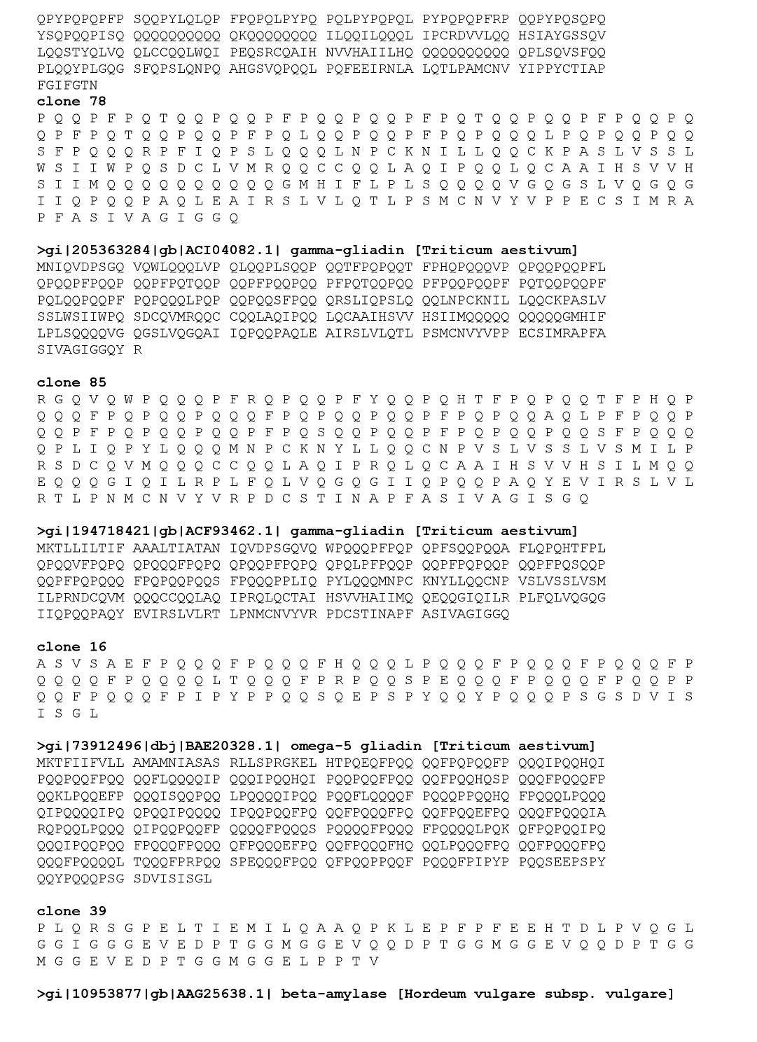

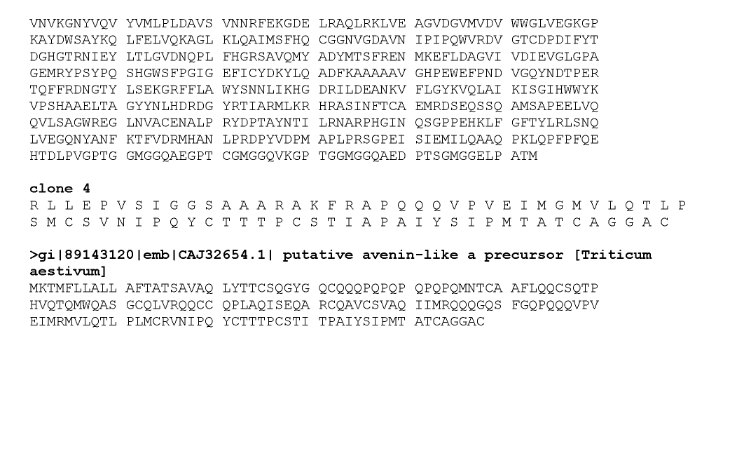

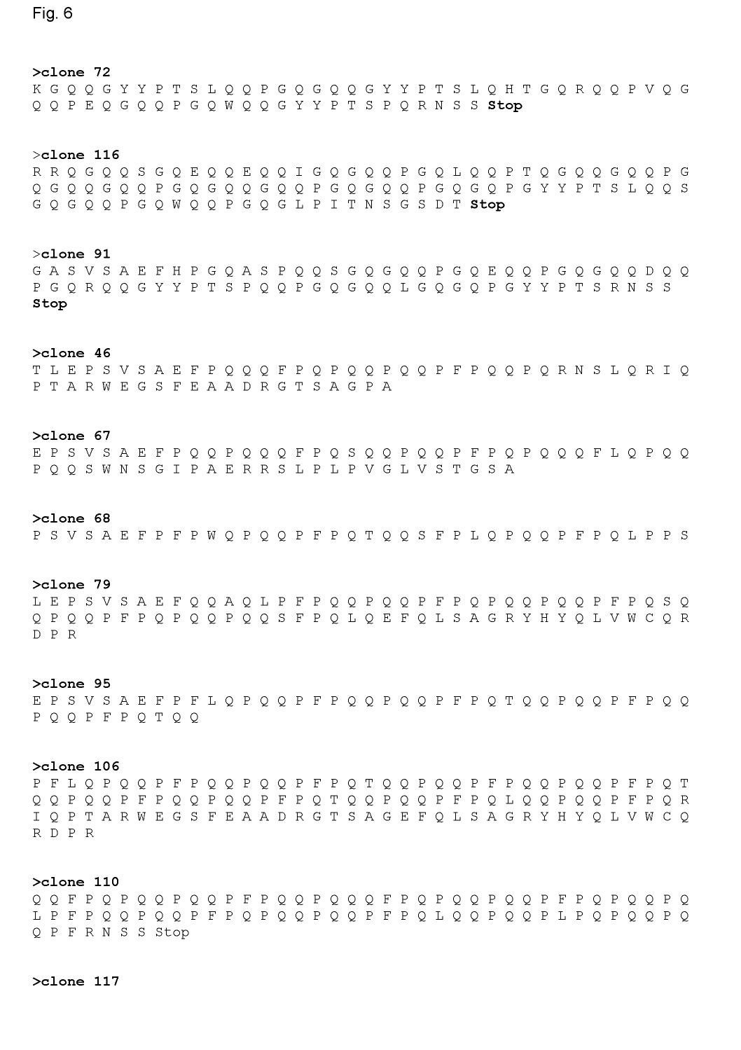

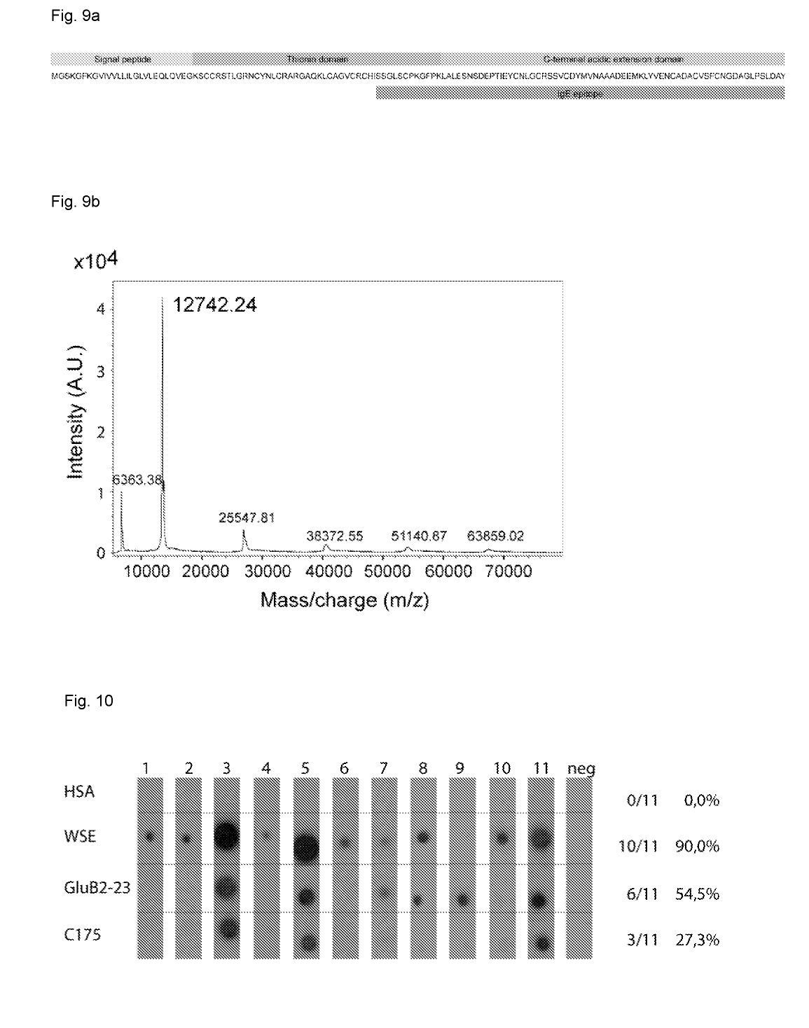

The comparison of the deduced amino acid sequences of the IgE-reactive phage clone 84 with published sequences showed that it is a wheat α-purothionin. The structural gene of α-purothionin includes regions encoding a typical signal peptide, a thionin domain (5 kDa) and a C-terminal acidic extension. The isolated nucleotide sequence of the clone 84-derived allergen (FIG. 4) shows IgE-reactivity, and this IgE epitope was related to the C-terminal acidic extension domain and a part of the thionin domain (FIG. 9a). The deduced amino acid sequence for the clone 84-derived allergen has a calculated molecular weight of 12.7 kDa and an isoelectric point (pI) of 6.27. The results of mass spectrometry analysis of purified recombinant protein corresponded with the deduced molecular weight of 12742 Da (FIG. 9b). The purity of the proteins was checked by 14% SDS-PAGE and Coomassie Blue staining (Fling, Bradford) and their identity was confirmed by Western blotting using a monoclonal anti-His tag antibody (Novagen). A Coomassie brilliant blue-stained 14% SDS-PAGE demonstrated the purity and migration of the recombinant allergen α-purothionin at 18 kDa.

### Example 4

### IgE Reactivity of the Recombinant Proteins

GluB3-23 is a major allergen in wheat dependent food allergy IgE reactivity of wheat food allergic patients to GluB3-23 and C175 was tested by dot blot analysis shown in FIG. 10 and FIG. 11. 0.5 μg of purified recombinant proteins (GluB3-23 and C175), 2 μg aqueous wheat seed (WSE) extract and 0.5 μg of human serum albumin (HSA) were dotted onto nitrocellulose (Whatman Protran nitrocellulose membrane, Sigma Aldrich) strips and after blocking with buffer A (50 mM sodium phosphate buffer, pH7.4, 0.5% w/v BSA, 0.5% v/v Tween-20, 0.05% w/v NaN₃) incubated with 1:10 diluted sera from patients suffering from wheat food allergy. Bound IgE Abs were detected with 1:10 diluted ¹²⁵I labeled anti human IgE Abs and visualized by autoradiography.

Sera were obtained from populations of patients suffering from wheat food allergy. Patients were selected according to positive case history, positive Skin Prick Test (SPT), double blind or open food challenge or CAP-test (Phadia, Uppsala, Sweden) to wheat.

In FIG. 10 it is demonstrated that 27.3% of the patients show IgE reactivity to C175 and 54.5% of these populations show IgE reactivity to the full length protein GluB3-23. In FIG. 11 it is demonstrated that 73.1% of another population of patients show IgE reactivity to C175 and 80.8% show IgE reactivity to the full length allergen GluB3-23. According to WHO/IUIS Allergen Standardization Committee definition (www.allergen.org) a major allergen has to be recognized by 50% of patients. Therefore, LMW GluB3-23 is a major allergen in wheat food allergy and a promising allergen for diagnosis and possibly for therapy.

Furthermore we showed that most of the epitopes for IgE recognition are localized on the N-terminal part of GluB3-23. An inhibition dot blot was performed with patients' sera. The sera were pre incubated with 10 μg of recombinant GluB3-23, C175 or Bet v 1. Bound IgE Abs were detected with ¹²⁵I-labelled anti human IgE Abs and visualized by autoradiography, the dot intensity was measured by a gamma counter. Table 2 shows the inhibition calculated in percentage which demonstrates that the C-terminal part of GluB3-23, C175 has a low potential to inhibit IgE binding to GluB3-23 binding.

### Glu-B1 is at Least a Minor Allergen in Wheat Dependent Food Allergy

The recombinant high molecular weight proteins mal43 and mal82, representing partial proteins of Glu-B1, were tested in dot blots with sera from patients from a population shown in FIG. 11. Sera were obtained from patients suffering from wheat food allergy. Patients were selected according to positive case history and positive Skin Prick Test (SPT) or CAP-test (Phadia, Uppsala, Sweden) to wheat. 0.5 μg of purified recombinant proteins (mal43 and mal82), 2 μg aqueous wheat seed (WSE) extract and 0.5 μg of human serum albumin (HSA) were dotted onto nitrocellulose (Whatman Protran nitrocellulose membrane, Sigma Aldrich) strips and after blocking with buffer A incubated with 1:10 diluted patients sera. Bound IgE Abs were detected with 1:10 diluted ¹²⁵I labeled anti human IgE Abs and visualized by autoradiography. 30.8% of the wheat food allergic patients showed IgE reactivity to these allergens (FIG. 11). On the basis of WHO/IRIS Allergen Standardization Committee definition (www.allergen.org), an allergen recognized by 10% of the patients is a minor allergen.

### α-Purothionin

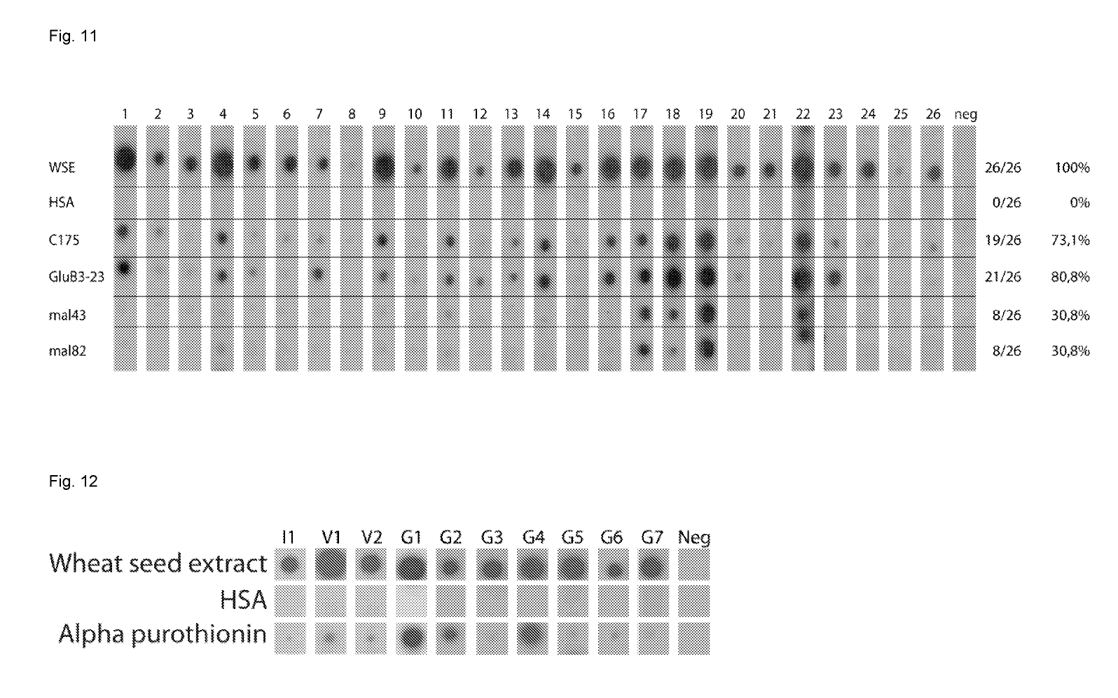

The IgE reactivity of dot-blotted recombinant wheat α-purothionin was tested with serum IgE antibodies from patients suffering from wheat food allergy (Table 3). Each of these patients, showing IgE-reactivity to α-purothionin exhibited IgE reactivity to dot-blotted wheat seed extract. 23% of patients from one population (n=13) and 29% of the patients from another population (n=24) reacted to the recombinant α-purothionin. (FIG. 12).

### Example 5

### Sequence Alignments and Different Crop Extracts

### GluB3-23 and C175

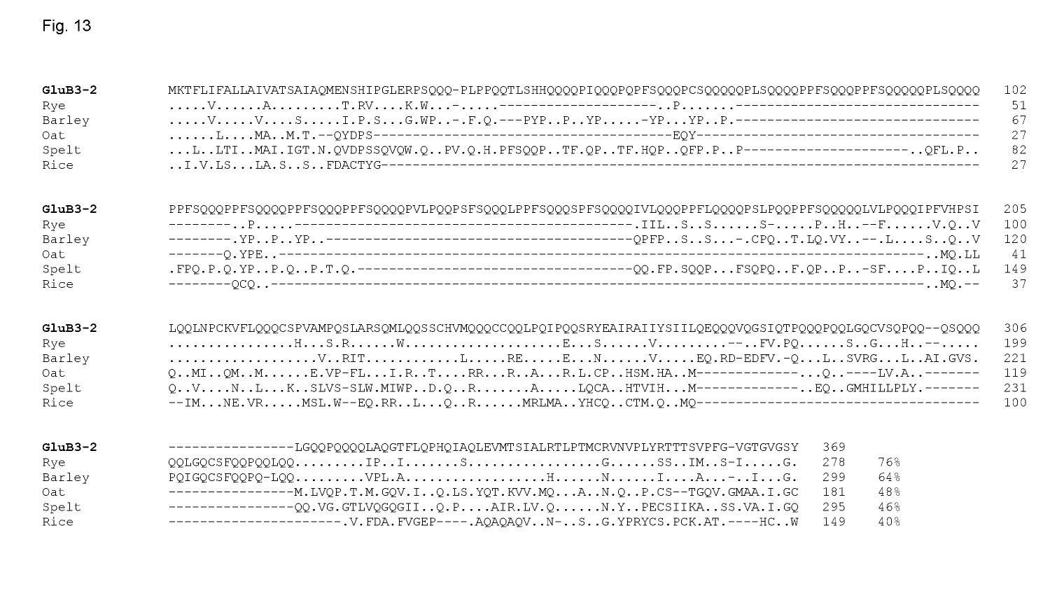

In order to find out if the GluB3-23 wheat allergen has homologues in other crop sorts, an amino acid sequence alignment with rye (Secale sylvestre), barley (Hordeum brevisubulatum), oat (Avena sativa), spelt (Triticum aestivum subsp. Spelta), and rice (Oryza sativa) was performed shown in FIG. 13. Rye shows an identity of 76%, barley 64%, oat 48%, spelt 46% and rice 40% to GluB3-23 in wheat (Triticum aestivum). The most conserved domains were the signal peptide and the C-terminal domains. Subsequently aqueous extracts of the different crops were prepared. 15 grams of the crop were homogenized, 32 ml H₂O and 32 μA Phenylmethylsulfonylfluorid (PMSF) were added and stirred for 4 hours at 4° C. The extracts were centrifuged to remove unsolvable particles. The aqueous extracts were loaded on a preparative 12.5% SDS PAGE and a protein molecular weight marker (PageRuler Plus; Prestained Protein Ladder, Fermentas) was used as standard and proteins were blotted onto nitrocellulose membrane (Whatman Protran nitrocellulose membrane, Sigma Aldrich). The membranes were blocked in Buffer A (50 mM sodium phosphate buffer, pH 7.4, 0.5% w/v BSA, 0.5% v/v Tween-20, 0.05% w/v NaN₃) after that they were incubated over night with rabbit preimmune serum or with rabbit Abs raised against C175 or GluB3-23 diluted 1:10000 in buffer A. Then sera were discarded and the membrane was washed three times with buffer A. The bound primary antibodies were detected with ¹²⁵I labelled anti rabbit IgG Abs (BSM diagnosic, Vienna, Austria), diluted 1:1000 in buffer A and visualized by Kodak XOMAT films with intensifying screens (Kodak, Heidelberg, Germany).

The membranes were incubated with the two different rabbit antibodies against C175 and GluB3-23 in order to identify the cross-reactive parts of the allergen. The bound C175 Abs and GluB3-23 Abs, respectively, were detected by ¹²⁵I labeled anti rabbit IgG Abs and visualized by autoradiography. GluB3-23 homologues could be detected in all extracts whereas the C175 antibody was not able to detect homologues in oat, and in all other extracts the reaction was weaker than with GluB3-23 antibodies.

### α-Purothionin

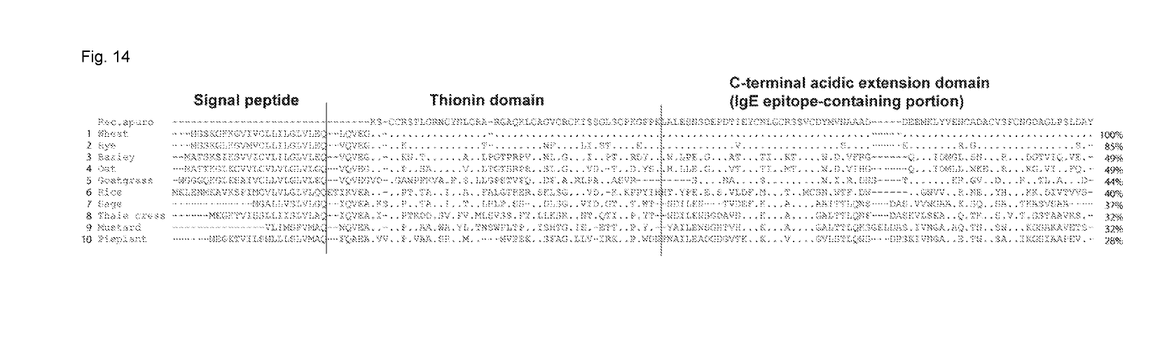

In order to study the cross-reactivity of the clone 84-derived allergen, a multiple sequence alignment of the clone 84-derived allergen α-purothionin with homologous proteins in other plants showed that the protein is also very common in other plant species. The amino acid sequence (single letter code) of clone 84-derived α-purothionin was aligned with purothionins in wheat (gi|4007850), rye (gi|4007745), barley (gi|246215), oat (gi|21069045), goatgrass (gi|1052551), rice (gi|215768993), sage (gi|77543393), thale cress (gi|21553588), mustard (gi|20564556), pieplant (gi|197312881) (FIG. 14). The clone 84-derived allergen α-purothionin shares the highest degree of sequence identity with α-purothionins from rye (85%), barley (49%) and also exhibits sequence identities of more than 30% with α-purothionins from several other plant sources (e.g., oat 49%, goatgrass 44%, rice 40%, sage 37%). Nitrocellulose-blotted extracts were probed with rabbit antibodies specific for alpha purothionin and for control purposes, with the corresponding pre-immune serum. α-purothionin specific antibodies were shown to detect the allergen in SDS-protein extracts from other plant species mentioned above like rye and barley.

### Example 6

### Protein Expression During Wheat Seed Maturation

### GluB3-23 and C175

Wheat (Triticum aestivum) seed SDS extracts were prepared 7, 10, 15, 20, 25, 30, 35 days after pollination and from mature wheat seeds according to Constantin et al (5). The extracts were separated by gel electrophoresis and blotted onto nitrocellulose membrane. The membrane was incubated with rabbit antibodies raised against GluB3-23. The bound anti GluB3-23 antibodies were detected with ¹²⁵I labeled anti rabbit IgG antibodies and visualized by autoradiography. It was clearly demonstrated that GluB3-23 accumulates in the wheat seed during maturation.

### Example 7

### In Vitro Digestion Assays

### GluB3-23 and C175

The stability of allergens in digestion assays indicates that a protein, parts of which are not totally digested and are detectable by protein-specific antibodies, belongs to the food allergens (7). Gastric and duodenal in vitro digestion was performed with aqueous wheat seed extracts as described previously (7); with the modification that for the duodenal digestion the commercial enzyme tablet Pankreoflat-Dragee (Solvay Pharma, Hannover, Germany) was used. The digested proteins were detected with rabbit antibodies raised against C175 and GluB3-23.

In gastric as well as in duodenal digestion assays it was demonstrated that only the anti GluB3-23 antibody was able to detect bands after digestion. The C175 part of GluB3-23 was digested in gastric digestion assay after 5 minutes, whereas GluB3-23 N-terminal parts could be detected after 120 minutes digestion. The duodenal digestion assay resulted in a similar pattern. C175 fragments could not be detected after 2 minutes of digestion but parts of GluB3-23 could be detected after 45 minutes of duodenal digestion. Consequently, it was demonstrated, that the N-terminal part of GluB3-23 is the stable indigestible fragment.

### Example 8

Gliadins were extracted from wheat grains with 70% ethanol following the Weiss et al (8) procedure. The extracted gliadins were then solubilised by dialysing against Buffer A containing 50 mM Tris buffer pH 4.0 and 4 M urea. The solubilised gliadin was passed through Sulfopropyl (SP) sepharose equilibrated with Buffer A, connected to the FPLC (Fast Protein Liquid Chromatography) machine. The flow through fractions were collected and labelled as FT SP. The column was washed with Buffer A and the bound proteins in the SP column were eluted using Buffer B containing 50 mM Tris pH 4.0, 4 M urea and a salt gradient of 0-500 mM NaCl and these fractions were labelled as Elu SP. A part of the FT SP fraction was dialysed in Buffer C containing 50 mM Tris pH 10.0 and 4 M urea and passed through the Diethyl aminoethyl (DEAE) sepharose column equilibrated with the same buffer. The flow through fractions were collected and labelled as FT DEAE. The column washed with Buffer C and the bound proteins were eluted with Buffer C containing a gradient of 0-500 mM NaCl. The elution fractions were labelled as Elu DEAE.

### Example 9

### Identification of Celiac Disease Specific Wheat Protein and Peptide Antigens

Whole wheat extract, whole gliadins and the four fractions FT SP, Elu SP, FT DEAE and Elu DEAE, obtained as described in Example 8 above, were separated by single dimension reducing SDS gel electrophoresis and blotted onto nitrocellulose membrane and probed with serum IgA from well characterized celiac and non celiac patients. Proteins in the FT SP and FT DEAE fractions were apparently highly specific for the disease since the non-celiac patients and patients on wheat free diet showed less reactivity to the proteins in the FT SP and FT DEAE fractions but were more reactive to other fractions and in general to the whole wheat extract and whole gliadin extract (data not shown).

### Example 10

### Specificity to Celiac Disease

Whole wheat extract, gliadins, the four fractions of gliadins, aqueous soluble wheat proteins and the SDS soluble glutenins were separated by electrophoresis and blotted onto nitrocellulose membrane and probed with antibodies generated in rabbit against a clone identified to be involved in wheat allergy. The FT fractions (FT SP and FT DEAE) had significantly better specificity for celiac disease, i.e. most of the proteins in this fraction showed positive IgA reactivity only to CD patients' sera and not to sera of CD patients on gluten free diet and healthy controls, whereas the Elu fractions (Elu SP and Elu DEAE) contained material which also showed IgA reactivity with sera from healthy control persons or celiac disease patients on diet (data not shown).

### Example 11

### Identification of Peptides and Proteins by Mass Spectrometry

The four fractions were digested with Pepsin/trypsin enzyme mixture according to standard protocols and the peptides obtained were identified by ESI-LC/MS mass spectrometry (HCT ULTRA from Bruker Daltonics). It was found that gamma gliadins were enriched in the FT SP fraction and in the FT DEAE fraction. All the peptides obtained are shown in Table 4a (SEQ ID NO:s 62-86 and 108-110).

### Example 12

### Expression and Purification of Recombinant Gamma Gliadin 1 and Gamma Gliadin 2

Gamma gliadin 1 sequence (SEQ ID NO: 87) and gamma gliadin 2 (SEQ ID NO: 88) (Table 4b) were cloned into pET27b E. coli expression vector to be expressed as recombinant proteins containing additional 6×Histidine residues at its C terminal end. The pET 27b-GG1 and the pET 27b-GG2 constructs were transformed into E. coli BL21 (DE3). The transformed cells were grown in 1 liter Luria Broth medium containing 50 mg/l Kanamycin at 37° C. The cells were grown until an OD₆₀₀ of 0.6, and the protein expression was induced by addition of isopropyl β-D-thiogalactopyranoside (IPTG) to a final concentration of 0.5 mM and incubation for 12 hours at 30° C. The cells were harvested by centrifugation and resuspended in lysis buffer (50 mM Tris pH 8.0, 500 mM NaCl, 10% glycerol) and PMSF (phenylmethanesulfonylfluoride) was added shortly before lysis. The cells were then lysed using ULTRA-TURRAX (IKA) disperser and the suspension was centrifuged at high speed to extract the inclusion bodies. The pellet containing the recombinant proteins as inclusion bodies was solubilized in 8M urea buffer and the 6×Histidine tagged GG1 and GG2 purified under denaturing conditions using Ni-Nta chromatography performed according to QIAexpressionist handbook (QIAGEN, Hilden, Germany). The fractions containing the purified proteins were dialyzed stepwise against 50 mM Tris pH 8.0, 100 mM NaCl, 10% glycerol buffer to remove urea and stored as aliquots in −20° C. The protein concentration was determined with a BCA Assay Kit (Novagen).

### Example 13

### IgA Reactivity to Wheat Food Antigens

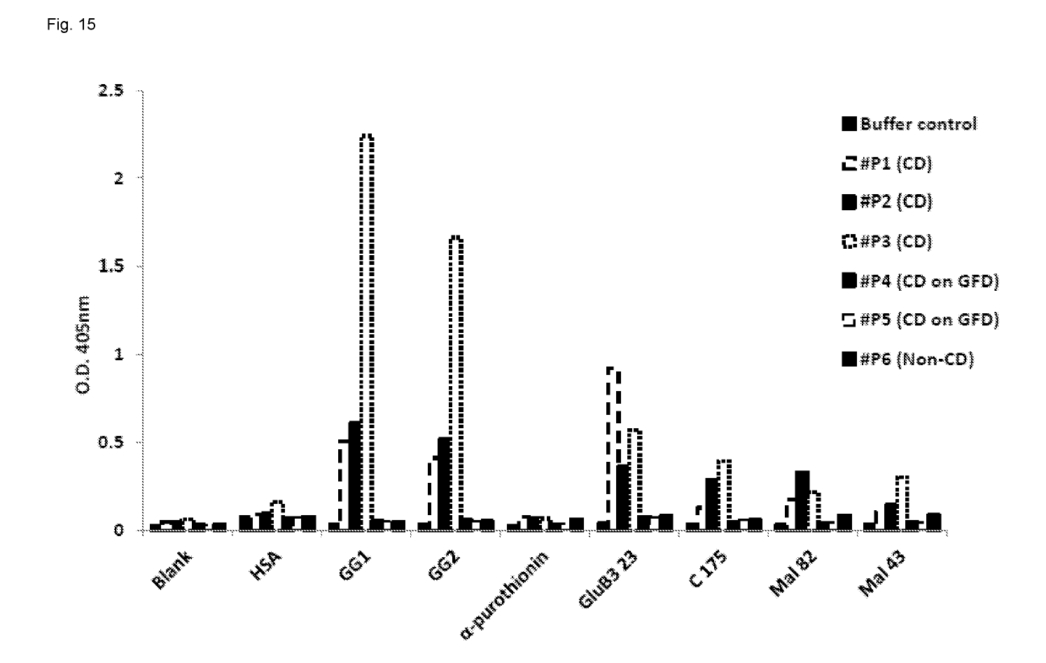

100 μl of the wheat proteins GG1 (Gamma gliadin 1), GG2 (Gamma gliadin 2), α-purothionin, GluB3-23, C-175, Mal 82 and Mal 43 of a concentration of 5 μg/ml were coated overnight at 4° C. onto Nunc Maxisorp Elisa plates. The remaining free binding sites were blocked with 1% BSA in PBST for 2 hours at room temperature. 100 μl of sera from celiac disease patients, celiac patients on diet and negative controls were added at a dilution of 1:100 in 0.5% BSA in PBST buffer and incubated for 12 hours at 4° C. The plates were washed 5 times in PBST buffer and 100 μl of mouse anti-human IgA₁/A₂ (BD Biosciences) diluted to 1:1000 in 0.5% BSA/PBST buffer was added and incubated at room temperature for 5 hours. The plates were then washed 5 times with PBST and 100 μl of sheep anti-mouse IgG conjugated with Horse radish peroxidise (HRP) (Amersham) antibodies were added and incubated at 37° C. for one hour. The plates were washed 3 times with PBST and the antibodies were detected using the HRP-ABTS(2,2′-azino-bis(3-ethylbenzothiazoline-6-sulfonate) detection system and colour reaction was measured at 405 nm in an ELSA plate reader. The values generated are plotted as a graph, see FIG. 15. Recombinant GG1 and GG2 show high IgA reactivity, α-puthionin shows negative IgA reactivity, and the LMW glutenin GluB3-23 and C-175 and HMW-glutenins (Mal 82 and Mal 43) show moderate IgA reactivity. The results show that the recombinant proteins GG1 and GG2 are highly specific for IgA reactivity. Absence of IgA reactivity to α-purothionin shows its specificity as an IgE binding allergen. Hence it is a very specific candidate protein for IgE reactivity.

### Example 14

### Synthesis of Overlapping Gamma Gliadin Peptides for Mapping T Cell and B Cell Epitopes

19 GG1 peptides were synthesized by FMOC technique, spanning the entire length of the GG1 protein and overlapping each other by five amino acid residues, and each peptide being 18-26 amino acid residues in length (Table 5, SEQ ID NO: 89-107). After synthesis, the peptides were purified by HPLC using gradient of 0-100% Acetonitrile with 0.1% TFA. The purified peptides were analyzed by MALDI for correct molecular mass and the pure fractions were freeze dried and stored at −20° C. until further use.

### Example 15

### IgA Reactivity of Celiac Disease Patients to Synthetic GG1 Peptides

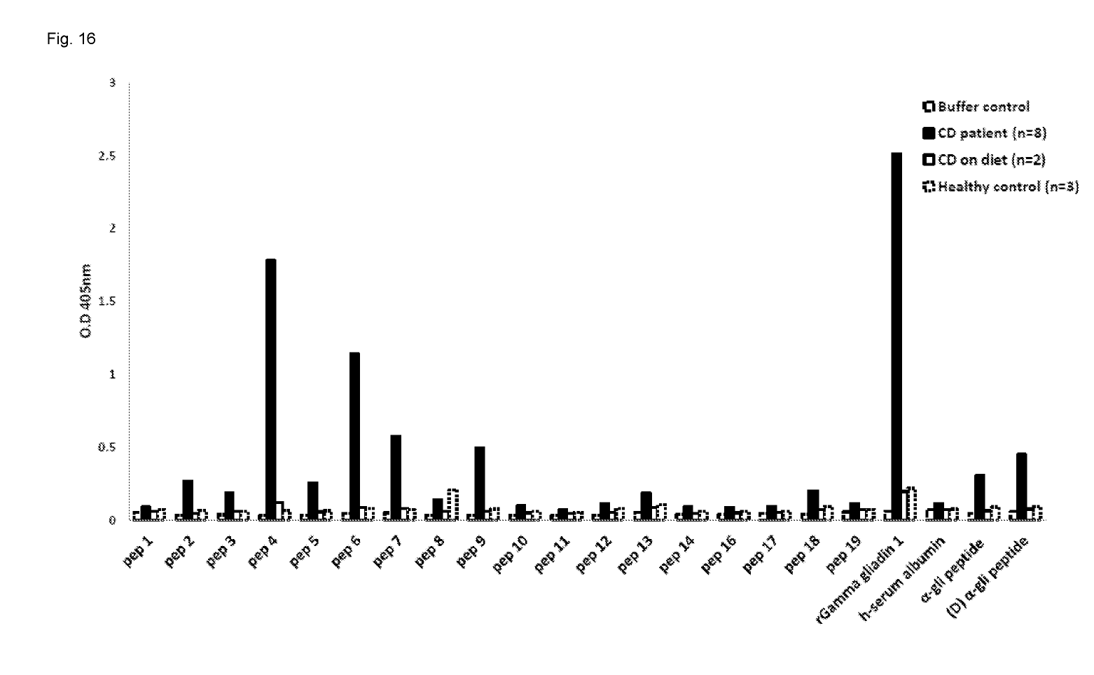

ELISA plates were coated with 100 ul of 5 μg/ml of the 19 overlapping peptides, rGG1, alpha-gliadin peptide p56-75 and deamidated alpha gliadin peptide p56-75 (Q65E) and probed with serum-IgA from CD patients, CD patients on gluten free diet and healthy controls and detected using mouse anti-human IgA1/A2 and sheep anti-mouse IgG-HRP labelled antibody. Human serum albumin (HSA) was used as control (n=8 CD patients, 2 CD patients on gluten free diet and 3 healthy controls). Serum IgA reactivity from celiac disease patients showed that the N-terminal region, rich in proline and glutamines had higher IgA reactivity than the c terminal region of the protein, which is poor in proline and glutamine (FIG. 16). Each of the synthetic GG1 peptides no. 2-7, 9, 13 and 18 (i.e. SEQ ID NO: 90-95, 97, 101 and 106, respectively) showed specific IgA reactivity. Comparison of IgA reactivity of the GG1 peptides with a known immunodominant epitope alpha gliadin P56-75 and deamidated alpha-gliadin peptide P56-75 (Q65E) showed that peptide 4 (SEQ ID NO: 92) and peptide 6 (SEQ ID NO: 94) had better sensitivity, as well as peptide 7 (SEQ ID NO: 95) and peptide 9 (SEQ ID NO: 97).

### Example 16

### IgG (Total) Reactivity of Celiac Disease Patients to Synthetic GG1 Peptides

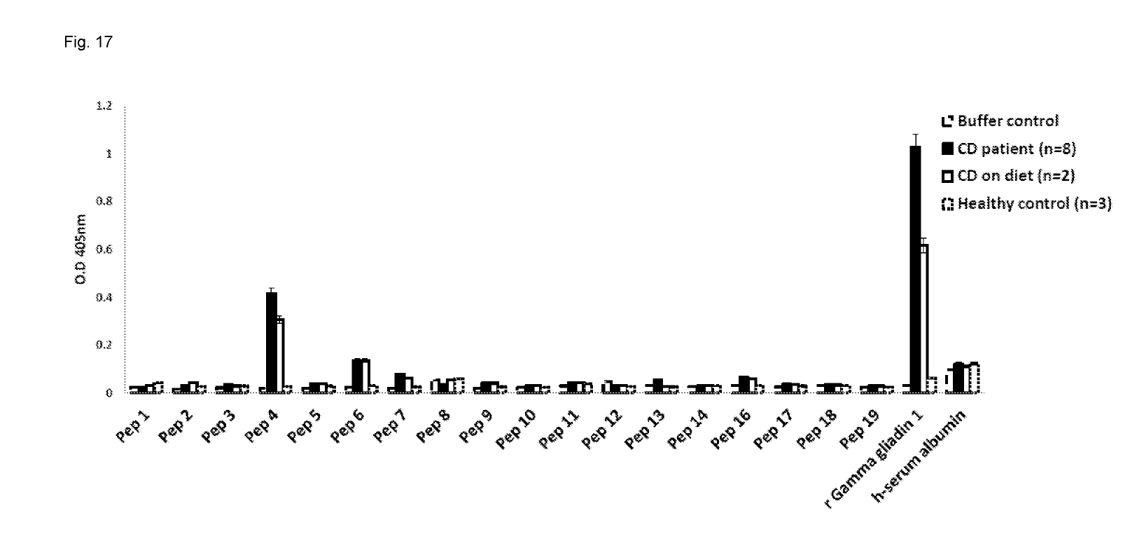

ELISA plates were coated with 100 ul of 5 μg/ml of the 19 overlapping synthetic peptides and rGG1 and probed with serum from CD patients, CD patients on gluten free diet and healthy controls and detected using mouse anti-human IgG (total) and sheep anti-mouse IgG-HRP labelled antibody. Human serum albumin (HSA) was used as control (n=8 CD patients, 2 CD patients on gluten free diet and 3 healthy controls). Reactivity to peptides in the N terminal region was higher than the C terminal region. The sensitivity and specificity of the assay using IgG was low (FIG. 17).

The celiac disease specific proteins and peptides disclosed in the above examples 14-16 are useful for antibody-based diagnosis, both IgA and IgG testing, preferably IgA-based testing.

### Example 17

### Dermatitis Herpetiformis Patient's Serum IgA Reactivity to Recombinant Gamma Gliadins

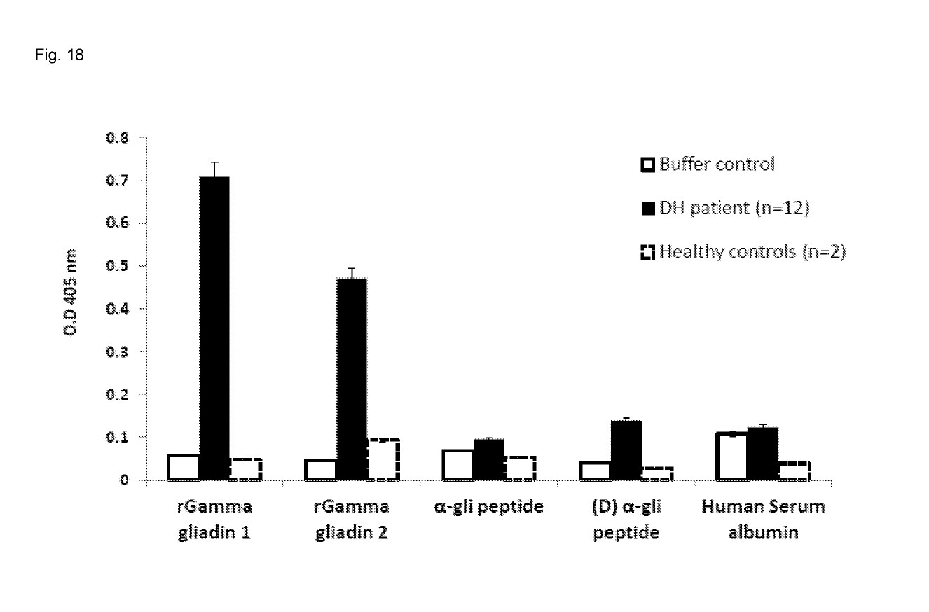

ELISA plates were coated with 100 μl of 5 μg/ml rGamma gliadin 1, rGamma gliadin 2, α-gliadin peptide p56-75 (α-gli peptide), deamidated α-gliadin peptide (D) and Human serum albumin. The bound proteins were probed with sera from Dermatitis herpetiformis (DH) patients and healthy controls. IgA reactivity to the proteins was detected using mouse anti-human IgA1/A2 and sheep anti-mouse IgG-HRP labelled antibody. (n=12 DH patients and 2 healthy controls). Recombinant GG1 and GG2 showed higher IgA reactivity to sera from DH patients than to healthy controls (FIG. 18). This result suggests that recombinant proteins GG1 and GG2 can be used in the diagnosis of Dermatitis herpetiformis.

TABLE 1
IgE reactive clones and their corresponding proteins

| | | SEQ ID | | |
|---|---|---|---|---|
| | | NO of | | SEQ |
| | | corre- | | ID NO |
| cDNA | SEQ | sponding | | corre- |
| clone | ID | amino | | sponding |
| no. | NO | acid seq. | Corresponding protein | protein |
| 175 | 1 | 26 | >gi\|169666917\|gb\|ACA63857.1\| | 51 |
| | | | LMW glutenin subunit [Triticum | |
| | | | aestivum] | |
| 43 | 2 | 27 | >gi\|71084277\|gb\|AAZ23584.1\| | 52 |
| | | | HMW glutenin x-type subunit Bx7 | |
| | | | precursor [Triticum aestivum] | |
| 82 | 3 | 28 | >gi\|71084277\|gb\|AAZ23584.1\| | 52 |
| | | | HMW glutenin x-type subunit Bx7 | |
| | | | precursor [Triticum aestivum] | |
| 84 | 4 | 29 | >gi\|4007850\|emb\|CAA65313.1\| | 53 |
| | | | alpha purothionin [Triticum | |
| | | | aestivum] | |
| 50 | 5 | 30 | >gi\|94315063\|gb\|ABF14401.1\| 1Dx | 54 |
| | | | high molecular weight glutenin | |
| | | | subunit [Triticum aestivum] | |
| 118 | 6 | 31 | >gi\|6684164\|gb\|AAF23507.1\| | 55 |
| | | | AF216869_1 glutenin, high | |
| | | | molecular weight subunit type y | |
| | | | precursor [Triticum aestivum] | |
| 34 | 7 | 32 | >gi\|154268818\|gb\|ABS72146.1\| | 56 |
| | | | alpha gliadin [Triticum aestivum] | |
| 78 | 8 | 33 | >gi\|205363284\|gb\|ACI04082.1\| | 57 |
| | | | gamma-gliadin [Triticum aestivum] | |
| 85 | 9 | 34 | >gi\|194718421\|gb\|ACF93462.1\| | 58 |
| | | | gamma-gliadin [Triticum aestivum] | |
| 16 | 10 | 35 | >gi\|73912496\|dbj\|BAE20328.1\| | 59 |
| | | | omega-5 gliadin [Triticum aestivum] | |
| 39 | 11 | 36 | >gi\|10953877\|gb\|AAG25638.1\| | 60 |
| | | | beta-amylase [Hordeum vulgare | |
| | | | subsp. vulgare] | |
| 4 | 12 | 37 | >gi\|89143120\|emb\|CAJ32654.1\| | 61 |
| | | | putative avenin-like a precursor | |
| | | | [Triticum aestivum] | |
| 46 | 13 | 38 | IgE reactive epitope | |
| 67 | 14 | 39 | IgE reactive epitope | |
| 68 | 15 | 40 | IgE reactive epitope | |
| 79 | 16 | 41 | IgE reactive epitope | |
| 95 | 17 | 42 | IgE reactive epitope | |
| 106 | 18 | 43 | IgE reactive epitope | |
| 110 | 19 | 44 | IgE reactive epitope | |
| 117 | 20 | 45 | IgE reactive epitope | |
| 190 | 21 | 46 | IgE reactive epitope | |
| 195 | 22 | 47 | IgE reactive epitope | |
| 72 | 23 | 48 | IgE reactive epitope | |
| 116 | 24 | 49 | IgE reactive epitope | |
| 91 | 25 | 50 | IgE reactive epitope | |

TABLE 2
IgE inhibition

| Pre- | Bet v 1 | | GluB3-23 | | C175 | |
|---|---|---|---|---|---|---|
| incubation | cpm | % inhibition | cpm | % inhibition | cpm | % inhibition |
| Pat 8 | 75.0 | 0.00 | 31.5 | 100.0 | 73.9 | 3.0 |
| Pat 10 | 377.6 | 0.00, 0 | 36.0 | 100.0 | 379.1 | 0.0 |
| Pat 3 | 829.0 | 0.00, 0 | 326.0 | 63.3 | 650.0 | 22.6 |
| Pat 11 | 379.0 | 0.00, 0 | 58.9 | 93.8 | 190.0 | 55.3 |
| Pat 12 | 11.5 | — | 17.7 | — | 16.2 | — |

TABLE 3
Demographic, clinical and serological characterization of patients
with wheat-dependent food allergy

| | | | | Total | | |
|---|---|---|---|---|---|---|
| | | | | IgE | Wheat-specific IgE | |
| Patient | Age | Sex | Symptoms | (kU/L) | (kU/L) | Positive SPT results |
| I1 | 12 | F | A | 562 | 68.8 | w(4) |
| V1 | 41 | | GE, AS, D | 252 | >100 (6) | w, hdm, r, c |
| V2 | 19 | M | GE, AS | 653 | >100 (6) | w, r, gp, e, b, n |
| G1 | 14 | M | A, AD, AST | 636 | >200 | w(8), ew(9), ey(8) |
| G2 | 3 | M | A, AD | 165 | 11.7 | w(8) |
| G3 | 3 | M | A, AD | 349 | 8.09 | w(4), ew(4.5), ey(3.5) |
| G4 | 3 | F | U, AD, AST | 795 | 165 | w(7.5), ew(6.5), ey(4) |
| G5 | 5 | M | A, AD, AST | 2723 | 695 | w(7) |
| G6 | 5 | M | AD, AST | 2724 | 48 | |
| G7 | 12 | F | U, AD, AST | 4539 | >100 | w(11) |

kU/L: kilounit per liter,
A: anaphylaxis,
AD: atopic dermatitis,
AS: airway symptoms,
AST: asthma,
D: dyspnea,
GE: gastroenteral symptoms,
U: urticaria,
b: birch,
c: cat,
e: egg,
ew: egg white,
ey: egg yolk,
gp: grass pollen,
hdm: house dust mite,
n: nut,
r: rye,
w: wheat

TABLE 4a
Peptides identified by mass spectrometry after peptic-tryptic digestion

| Fraction | SEQ ID NO | Peptide sequence | Class of gliadins |
|---|---|---|---|
| FT SP | 62 | AQIPQQLQ | γ-gliadins |
| | 63 | PQQQRPFIQPSL | γ-gliadins |
| | 64 | LVQGQGIIQPQQPAQLE | γ-gliadins |
| | 65 | APFASIVAGIGGQ | γ-gliadins |
| | 69 | LVPLSQQQQVGQGILV | γ-gliadins |
| | 70 | LPLYQQQQVGQGTLV | γ-gliadins |
| | 71 | FLPLSQQQQVGQGSLV | γ-gliadins |
| | 80 | LQQPNIAHASSQVSQQSYQLL | γ-gliadins |
| | 108 | LSQQQQVGQGSLV | γ-gliadins |
| | 109 | LYQQQQVGQGTLV | γ-gliadins |
| Elu SP | 72 | LQLQPFPQPQLP | α/β gliadins |
| | 73 | FFQPSQQNPQAQGSFQPQQLPQFE | α/β gliadins |
| | 74 | FRPSQQNPQAQGSVQPQQLPQF | α/β gliadins |
| | 75 | RVPVPQLQPQNPSQQQPQKQ | α/β gliadins |
| | 78 | LQQHNIAHGSSQVLQ | α/β gliadins |
| | 76 | LQQHNIAHASSQVLQQSTYQLLQ | α/β gliadins |
| | 79 | MVRVPVPQLQ | α/β gliadins |
| | 77 | LQQHNIAHGSSQVLQQSTYQLV | α/β gliadins |
| | 85 | LPQQPPFSQQQQPILP | LMW glutenin subunit |
| | 84 | LPQQQIPFVHPSILQ | LMW glutenin subunit |
| | 81 | FLQPHQIAQLE | LMW glutenins |
| | 82 | LAQGTFLQPHQIAQLE | LMW glutenins |
| | 83 | FSQQQQLFPQQPSFS | LMW glutenins |
| | 86 | LLQQQIPIVHPSILQ | LMW glutenin subunit |
| | 68 | LVQGQGIIQPQQPAQLE | γ-gliadins |
| FT | 65 | APFASIVAGIGGQ | γ-gliadins |
| DEAE | 66 | NIQVDPSGQVQALQ | γ-gliadins |
| | 67 | NIQVDPSGQVQWLQQ | γ-gliadins |
| | 68 | LVQGQGIIQPQQPAQLE | γ-gliadins |
| Elu | 79 | MVRVPVPQLQ | α/β gliadins |
| DEAE | 110 | LQQHSIAYGSSQVLQ | α gliadins |
| | 70 | LPLYQQQQVGQGTLV | γ-gliadins |
| | 80 | LQQPNIAHASSQVSQQSYQLL | γ-gliadins |

TABLE 4b
Gamma gliadin 1 (GG1) and gamma gliadin 2 (GG2)

| Protein | SEQ ID NO |
|---|---|
| Gamma gliadin 1 (GG1) | 87 |
| Gamma gliadin 2 (GG2) | 88 |

TABLE 5
Synthesized GG1 peptides

| Peptide no. | SEQ ID NO | rGG1 peptide sequence |
|---|---|---|
| 1 | 89 | MNIQVDPSGQVQWLQQQLV |
| 2 | 90 | QQQLVPQLQQPLSQQPQQTF |
| 3 | 91 | QQPQQTFPQPQQTFPHQPQQQ |
| 4 | 92 | QPQQQVPQPQQPQQPFLQPQQPFPQQ |
| 5 | 93 | PFPQQPQQPFPQTQQPQQ |
| 6 | 94 | QQPQQPFPQQPQQPFPQTQQ |
| 7 | 95 | PFPQTQQPQQPFPQLQQPQQ |
| 8 | 96 | QQPQQPFPQPQQQLPQPQQ |
| 9 | 97 | PQPQQPQQSFPQQQRPFI |
| 10 | 98 | QRPFIQPSLQQQLNPCKNIL |
| 11 | 99 | CKNILLQQSKPASLVSSLWS |
| 12 | 100 | LVSSLWSIIWPQSDCQVMRQ |
| 13 | 101 | QVMRQQCCQQLAQIPQQLQCA |
| 14 | 102 | QLQCAAIHSVVHSIIMQQQQQ |
| 15 | 103 | QQQQQQQQQQGIDIFLPLSQ |
| 16 | 104 | LPLSQHEQVGQGSLVQGQGI |
| 17 | 105 | QGQGIIQPQQPAQLEAIRSLV |
| 18 | 106 | IRSLVLQTLPSMCNVYVPPECS |
| 19 | 107 | PPECSIMRAPFASIVAGIGGQ |

### REFERENCES

1. Constantin, C., S. Quirce, M. Poorafshar, A. Touraev, B. Niggemann, A. Mari, C. Ebner, H. Akerstrom, E. Heberle-Bors, M. Nystrand, and R. Valenta. 2009. Micro-arrayed wheat seed and grass pollen allergens for component-resolved diagnosis. Allergy 64:1030-1037.

2. Green P H, Cellier C. Celiac disease. N Engl J Med. 2007;357(17):1731-43.

3. Sollid L M. Celiac disease: dissecting a complex inflammatory disorder. Nat Rev Immunol 2002;2(9):647-55.

4. Mitea, C., Y. Kooy-Winkelaar, P. van Veelen, A. de Ru, J. W. Drijfhout, F. Koning, and L. Dekking. 2008. Fine specificity of monoclonal antibodies against celiac disease-inducing peptides in the gluteome. Am J Clin Nutr 88:1057-1066.

5. Constantin, C., S. Quirce, M. Grote, A. Touraev, I. Swoboda, A. Stoecklinger, A. Mari, J. Thalhamer, E. Heberle-Bors, and R. Valenta. 2008. Molecular and immunological characterization of a wheat serine proteinase inhibitor as a novel allergen in baker's asthma. J Immunol 180:7451-7460.

6. Mothes-Luksch, N., S. Stumvoll, B. Linhart, M. Focke, M. T. Krauth, A. Hauswirth, P. Valent, P. Verdino, T. Pavkov, W. Keller, M. Grote, and R. Valenta. 2008. Disruption of allergenic activity of the major grass pollen allergen Phl p 2 by reassembly as a mosaic protein. J Immunol 181:4864-4873.

7. S. Vieths, J. R., U. Müller, A. Hoffmann, D. Haustein. 1998. Digestibility of peanut and hazelnut allergens investigated by a simple in vitro procedure. Eur Food Res Technol 209:379-388.

8. Weiss, W., C. Vogelmeier, and A. Gorg. 1993. Electrophoretic characterization of wheat grain allergens from different cultivars involved in bakers' asthma. Electrophoresis 14:805-816.

9. Clay, T. M., Hobeika, A. C., Mosca, P. J., Lyerly, H. K., and Morse, M. A. 2001. Assays for monitoring cellular immune responses to active immunotherapy of cancer. Clin Cancer Res 7: 1127.

10. Nilsen E. M., Lundin K. E., Krajci P, Scott H, Sollid L. M., Brandtzaeg P. 1995. Gluten specific, HLA-DQ restricted T cells from coeliac mucosa produce cytokines with Th1 or Th0 profile dominated by interferon gamma. Gut 37(6):766-76.

11. Reisinger J, Triendl A, Küchler E, Bohle B, Krauth M. T., Rauter I, Valent P, Koenig F, Valenta R, and Niederberger V. 2005. IFN-γ-enhanced allergen penetration across respiratory epithelium augments allergic inflammation. J Allergy Clin Immunol 115(5): 973-981.

12. Mittermann I, Reininger R, Zimmermann M, Gangl K, Reisinger J, Aichberger K. J., Greisenegger E. K., Niederberger V, Seipelt J, Bohle B, Kopp T, Akdis C. A., Spitzauer S, Valent P, and Valenta R. 2008. The IgE-Reactive Autoantigen Hom s 2 induces damage of respiratory epithelial cells and keratinocytes via induction of IFN-γ. J Investigative Dermatology 128, 1451-1459.

1. An isolated polypeptide comprising the amino acid sequence according to any one of SEQ ID NO: 26-50, 62-86, and 89-110.

2. The polypeptide according to claim 1, characterised in that it is isolated from wheat or recombinantly produced.

3. An isolated nucleic acid molecule encoding the polypeptide according to claim 1.

4. The nucleic acid according to claim 3 having the nucleotide sequence according to any one of SEQ ID NO: 1-25.

5. The polypeptide according to claim 1 or a fragment or variant thereof sharing epitopes for antibodies with said polypeptide, for use in therapy or diagnosis.

6. The polypeptide according to claim 1 or a fragment or variant thereof sharing epitopes for antibodies with said polypeptide, for use in therapy or diagnosis of celiac disease, dermatitis herpetiformis, or IgE-mediated allergy.

7. An isolated polypeptide comprising the amino acid sequence according to any one of SEQ ID NO: 51-61, 87 and 88 or a fragment or variant thereof sharing epitopes for antibodies with said polypeptide, for use in therapy or diagnosis.

8.-9. (canceled)

10. A pharmaceutical composition comprising a polypeptide having the amino acid sequence according to any one of SEQ ID NO: 26-110, or a hypoallergenic form of said polypeptide that is modified to abrogate or attenuate its T cell-, IgA- or IgE-binding response, and optionally pharmaceutically acceptable excipients, carriers, buffers and/or diluents.

11. A pharmaceutical composition according to claim 10, wherein said hypoallergenic form of said polypeptide is modified by fragmentation, truncation or tandemerization of the molecule, deletion of internal segments, domain rearrangment, substitution of amino acid residues, disruption of disulfide bridges.

12. A method for producing an allergen composition comprising the step of adding a polypeptide having the amino acid sequence according to any one of SEQ ID NO: 26-110, or a fragment or variant thereof sharing epitopes for antibodies with said polypeptide, to a composition comprising an allergen extract and/or at least one purified allergen component.

13. An allergen composition obtainable with the method according to claim 12.

14. A method for in vitro diagnosis of celiac disease comprising
contacting a body fluid or tissue sample from a mammal suspected of having celiac disease with at least one polypeptide having the amino acid sequence according to any one of SEQ ID NO: 62-110 or a fragment or variant thereof sharing epitopes for antibodies with said polypeptide; and
measuring the presence of activated T cells in the sample, such as by use of a lymphocyte proliferation assay, a FACS analysis of the cell activation, or by measuring cytokine release;
wherein the presence of activated T cells is indicative of celiac disease.

15. A method for in vitro diagnosis of celiac disease comprising
contacting leukocytes from a mammal suspected of having celiac disease with at least one polypeptide having the amino acid sequence according to any one of SEQ ID NO: 62-110 or a fragment or variant thereof sharing epitopes for antibodies with said polypeptide, in a medium;
contacting a cell sample from said mammal with the medium; and
measuring the presence of interferon-gamma or other cell-damaging substance(s) in the cell sample;
wherein the presence of interferon-gamma or other cell-damaging substance(s) is indicative of celiac disease.

16. The method according to claim 15, wherein the cell sample comprises intestinal epithelial cells.

17. A method for in vitro diagnosis of celiac disease, dermatitis herpetiformis, or IgE-mediated allergy, comprising
contacting a body fluid or tissue sample from a mammal suspected of having celiac disease, dermatitis herpetiformis, or IgE-mediated allergy with at least one polypeptide having the amino acid sequence according to any one of SEQ ID NO: 26-110 or a fragment or variant thereof sharing epitopes for antibodies with said polypeptide; and
detecting the presence, in the sample, of IgA or IgE antibodies specifically binding to said polypeptide or polypeptides;
wherein the presence of such antibodies specifically binding to said polypeptide or polypeptides is indicative of celiac disease, dermatitis herpetiformis, or IgE-mediated allergy.

18. A method according to claim 17, wherein the IgE-mediated allergy is wheat food allergy.

19. A diagnostic kit, comprising a polypeptide having the amino acid sequence according to any one of SEQ ID NO: 26-110 or a fragment or variant thereof sharing epitopes for antibodies with said polypeptide, or a composition according to claim 10.


SEQUENCE LISTING (110 sequences)

SEQ ID NO: 1 - DNA, 924 bp, Triticum aestivum
cgtcagcagctcggacaaggacaacaagggtactacccaacttctctgcagccaggacaaaagcagcaggcaggacaagggcaacaatcaggacaaggacaacaagggtactacccaacttccccgcaacagtcaggacaagggcaacaaccgggacaagggcaaccagggtactacccaacttctccgcagcagtcaggacaatggcagcaaccaggacaagggcaacaaccaggacaagggcaacaatcaggacaatggcaactagtgtactacccaacttctccacaacaacaaatagtactacagcaacgaccaccatttttgcaacaacaacaaccaagtctaccgcaacaaccaccattttcgcagcaacaacaacaactagttctaccgcaacaacaaataccatttgttcatccatctatcttgcagcagctaaacccatgcaaggtattcctccagcagcaatgcagccctgtggcaatgccacaaagtcttgctaggtcgcaaatgttgcagcagagcagttgccatgtgatgcaacaacaatgttgccagcagttgccgcaaatcccccagcaatcccgctatgaggcaatccgtgctatcatctactccatcatcctgcaagaacagcaacaggttcagggttccatccaaactcctcagcagcaaccccaacagttgggccaatgtgtttcccaaccccaacagcagtcacaacaacaactcgggcaacaacctcaacaacaacaattggcacaaggtacctttttgcagccacaccagatagctcagcttgaggtgatgacttccattgcgctccgtaccctgccaacgatgtgccgtgtcaatgtgccgttgtatagaaccaccactagtgtgccattcggtgttggcaccggagttggttcctactaa

SEQ ID NO: 2 - DNA, 842 bp, Triticum aestivum
cggaattccgggcaacaactgggacaagggcaaccagggtactacccaacttcgcagcagtcggaacaaaggcagcagccaggacaaggaaaacaaccaggacaaggacaacaagggtactacccaacttctccgcaacagtcaggacaagggcaacaactgggacaagggcaaccagggtactacccaacttctccacagcagtcaggacaaggacaacaatcaggacaaggacaacaagggtactacccaacttctccgcaacagtcaggacaagggcaacaaccgggacaagggcaatcggggtacttcccaacttctcggcagcagtcaggacaagggcagcagccaggacaaggacaacagtcgggacaagggcaacaaggtcagcaaccaggacaaggacaacaagcgtactacccaacttcttcgcaacagtcaagacaaaggcaacaggcaggacaatggcaacgaccgggacaagggcaaccagggtactacccaacctctccacagcagccaggacaagagcaacaatcaggacaagcgcaacaatcaggacaatggcaactagtgtactacccaacttctccgcaacagccaggccaattgcaacaaccagcacaagggcaacaaccagcacaagggcaacaatcagcacaagagcaacagccaggacaagcgcaacaatcaggacaatggcaactagtgtactacccaacttctccgcaacagccaggacaattgcaacaaccagcacaagggcaacaagggtactacccaacttctccacaacagtcaggacaagggcaacaagggtactacccaacttcttcccgcaa

SEQ ID NO: 3 - DNA, 588 bp, Triticum aestivum
ccaacttctccacaacagtcaggacaagggcaacaagggtactacccaacttctccgcaacagtcaggacaagggcaacaagggtactacccaacttctccgcaacagtcaggacaagggcagcagccaggacaaggacaacagccaagacaagggcaacaagggtactacccaatttctccgcagcagtcaggacaagggcaacaaccaggacaagggcaacaacgatactacccaacttctccgcagcagtcaggacaagggcaacaaccaggacatgagcaacagccaggacaatggctgcaaccaggacaagggcaacaagggtactatccaacttcttcacagcagtcaggacaagggcatcaatcaggacaagggcaacaagggtactacccaacttctctgtggcaaccaggacaagggcaacaaccaggacaagggcaacaaggctacgccagcccataccatgttagcgcggagtaccaggcggcccgcctaaaggtggcaaaggcgcagcagctcgcggcacagctgccggcaatgtgccggctggagggcagcgacgcattgtcgaccaggcagtga

SEQ ID NO: 4 - DNA, 234 bp, Triticum aestivum
ctctcaagtggcctaagctgccctaaaggcttccctaaattggcccttgagtccaactcagatgaaccagacaccattgagtactgcaacttgggatgtaggtcttccgtgtgtgactacatggtcaacgcagctgctgacgatgaagagatgaaactctatgtggaaaattgtgctgatgcttgtgtcagtttctgtaacggtgatgctggcctcccatcccttgatgcgtac

SEQ ID NO: 5 - DNA, 495 bp, Triticum aestivum
cggaattcccagcagccaggacaagggcaacaaggtcagcagccaggacaagggcagcaaccgggacaagggcagccatggtactacccaacttctccgcaggagtcaggacaagggcaacagccaggacaatggcaacaaccaggacaagggcaaccagggtactacctaacttctccgttgcagctaggacaagggcaacaagggtactacccaacttctctgcaacaaccaggacaagggcagcaaccaggacaatggcaacaatcgggacaagggcaacattggtactacccaacttctccgcagctgtcaggacaagggcaacggccaggacaatggctgcaaccaggacaagggcaacaagggtactacccaacttctccgcaacagccaggacaagggcaacaactaggacaatggctgcaaccaggacaagggcaacaagggtactacccaacttctctgcaacagacaggacaagggcagcaatca

SEQ ID NO: 6 - DNA, 300 bp, Triticum aestivum
ccaggacaatggctgcaaccaggacaagggcaacaagggtactatccaacttcttcacagcagtcaggacaagggcatcaatcaggacaagggcaacaagggtactacccaacttctctgtggcaaccaggacaagggcaacaaccaggacaagggcaacaaggctacgccagcccataccatgttagcgcggagtaccaggcggcccgcctaaaggtggcaaaggcgcagcagctcgcggcacagctgccggcaatgtgccggctggagggcagcgacgcattgtcgaccaggcagtga

SEQ ID NO: 7 - DNA, 258 bp, Triticum aestivum
caacaacaacaacaacaacaacaaccgtcgagccaggtctcctaccagcagcctcagcaacaatatccatcaggccagggcttcttccagccatctcagcaaaacccacaggcccagggctttgtccaacctcagcaactgccgcagttcgaggaaataaggaacctagcgctgcagacgctaccagcaatgtgcaatgtctacatccctccatattgctcgaccaccattgcgccatttggcatcatgagtactaac

SEQ ID NO: 8 - DNA, 720 bp, Triticum aestivum
ccacaacaaccattcccccagactcaacaaccacaacaaccatttccccagcaaccacaacaaccatttccccagactcaacaaccccaacaaccatttccccaacaaccacaacaaccattcccccagactcaacaaccccaacaaccatttccccagctccagcaaccacaacaaccttttccccagccccaacaacaattgccgcagccccaacaaccgcaacaatcattcccccaacaacaacggccattcattcaaccatctctacaacaacagttgaacccatgcaagaatatcctcttgcaacaatgcaaacctgcgtcattggtgtcatccctctggtcaataatctggccacaaagcgattgcctagtgatgcggcaacaatgctgccaacaactagcacagattcctcaacagctccagtgcgcagccatccatagcgtcgtgcattccatcatcatgcagcagcagcagcaacaacaacaacaacaaggcatgcatatctttctgccactatctcagcagcaacaggtgggtcaaggttctctagtccaaggccagggcatcatccaaccacaacaaccagctcaattggaggcgatcagatcattggtgttgcaaactcttccatccatgtgcaacgtgtatgtcccacctgagtgctccatcatgagggcaccatttgccagcatagtcgcgggcattggtggccaa

SEQ ID NO: 9 - DNA, 780 bp, Triticum aestivum
cgcggccaagtacaatggccacaacaacaaccattccgccagccccaacaaccattctaccagcaaccacaacacacatttccccaaccccaacaaacattcccccatcaaccacaacaacaatttccccagccccagcaaccacaacaacaatttccgcagccccagcaaccacaacaaccatttccccagccccaacaagcccaactaccatttccccaacaaccacaacaaccattcccccagcctcaacaaccccaacaaccatttccccagtcacagcaaccacaacaaccttttccccagccccaacaaccgcaacaatcattcccccagcaacaacaaccgttgattcagccatatctacaacaacagatgaacccttgcaagaattacctcttacagcaatgcaaccctgtgtcattggtgtcatccctcgtgtcaatgatcttgccacgaagtgattgccaggtgatgcagcaacaatgttgccaacaactagcacagattcctcgccagctccagtgtgcagccatccatagcgtcgtgcattccatcctcatgcagcaagaacaacaacaaggcatacagatcctccggccactgtttcagctcgtccaaggtcagggcatcatccaacctcaacaaccagctcaatatgaggtgatcaggtcattggtattgagaacccttccaaacatgtgcaacgtgtatgtccgacctgactgctccaccatcaacgcaccatttgccagcatagtcgccggcatcagtggacaa

SEQ ID NO: 10 - DNA, 333 bp, Triticum aestivum
ccccaacagcaattcccgcaacaacaattccaccaacaacaattaccacaacaacaatttccccaacaacaattcccccaacagcaattcccccaacaacaacagttcccccaacaacaacaattaacgcaacaacaattcccccggccacaacaatcccctgaacaacaacaattcccccaacaacaattcccccagcaaccaccacaacaattcccccaacaacaatttccaataccatacccaccccagcaatcacaagaaccttccccataccaacaatatccacaacaacaaccatctgggagcgacgttataagtatcagtggccta

SEQ ID NO: 11 - DNA, 288 bp, Triticum aestivum
cctttgcaaagatcagggccagaactgacgattgaaatgatcctacaagcagcgcagccaaaactggagccattcccctttgaagagcacaccgacctgccagttcaaggcctcggtggcatcggtggtggggaggttgaagaccccactggtggcatgggtggggaggttcaacaagaccccactggtggcatgggtggggaggttcaacaagaccccactggtggcatgggtggggaggttgaagaccctaccggtggcatgggtggggagctccctcccaccgtg

SEQ ID NO: 12 - DNA, 225 bp, Triticum aestivum
cgactcctggagcccgtcagtatcggcggctcagcagcagcaagggcaaagtttcgggcgcctcagcagcaggttccggttgagataatggggatggtgcttcagacccttccgtcgatgtgcagcgtaaacatcccacaatattgcaccaccaccccgtgcagcaccatcgcccccgccatctacagcatccccatgacagctacctgtgctggtggtgcctgc

SEQ ID NO: 13 - DNA, 62 bp, Triticum aestivum
ccacaacaacaatttccccagccccagcaaccacaacaaccatttccccagcaaccacaacg

SEQ ID NO: 14 - DNA, 104 bp, Triticum aestivum
cctcaacaaccccaacaacaatttccccagtcacagcaaccacaacaaccttttccccagccccaacaacaattcctgcagccccaacaaccgcaacaatcatg

SEQ ID NO: 15 - DNA, 93 bp, Triticum aestivum
ccatttccctggcaaccacaacaaccatttccccagacccaacaatctttccctctgcaaccacaacaaccattcccccagctaccccccagc

SEQ ID NO: 16 - DNA, 147 bp, Triticum aestivum
caacaagcccaactaccatttccccaacaaccacaacaaccattcccccagcctcaacaaccccaacaaccatttccccagtcacagcaaccacaacaaccttttccccagccccaacaaccgcaacaatcattcccccagttgcag

SEQ ID NO: 17 - DNA, 122 bp, Triticum aestivum
ccatttctccagccccaacaaccattcccccaacaaccacaacaaccattcccccagactcaacaaccacaacaaccatttccccagcaaccacaacaaccatttccccagactcaacaacc

SEQ ID NO: 18 - DNA, 226 bp, Triticum aestivum
ccatttctccagccccaacaaccattcccccaacaaccacaacaaccattcccccagactcaacaaccacaacaaccatttccccagcaaccacaacaaccatttccccagactcaacaaccccaacaaccatttccccaacaaccacaacaaccattcccccagactcaacaaccccaacaaccatttccccagctccagcaaccacaacaaccttttccgcaaa

SEQ ID NO: 19 - DNA, 239 bp, Triticum aestivum
caacaatttccccagccccaacaaccacaacaaccatttccccagcaaccacaacaacaatttccccagccccaacaaccacaacaaccatttccccagccccaacaaccccaactaccatttccgcaacaaccacaacaaccattcccccagcctcaacaaccccaacaaccatttccccagttacagcaaccacaacaacctttaccccagccccaacaaccgcaacaaccattccg

SEQ ID NO: 20 - DNA, 297 bp, Triticum aestivum
cctagcggccaagtacaatggctacaacaacaactagtcccccagctccaacagccattatcccagcaaccacaacaaacatttccccaacctcaacaaacattcccccatcaaccacaacaacaagttccccagcctcagcaaccacaacaaccatttctccagccccaacaaccattcccccaacaaccacaacaaccattcccccagactcaacaaccacaacaaccatttccccgccagctgagcgccggtcgctaccattaccagttggtctggtgtcaacgggatccgcga

SEQ ID NO: 21 - DNA, 114 bp, Triticum aestivum
ccaaaggattcagcccaccgcccgttgggaagggagcttcgaggcggccggccgcggcacgtcggccggaccggcttagccaatggcacgggcccttgggggcgcaagcgcccc

SEQ ID NO: 22 - DNA, 291 bp, Triticum aestivum
ccatttctccagccccaacaaccattcccccaacaaccacaacaaccattcccccagactcaacaaccacaacaaccatttccccagcaaccacaacaaccatttccccagactcaacaaccccaacaaccatttccccaacaaccacaacaaccattcccccagactcaacaaccccaacaaccatttccccagctccagcaaccacaacaaccttttccgcaaaggattcagcccaccgcccgtggggaagggagcttcgaggcggccgaccgcggcacttcggccggg

SEQ ID NO: 23 - DNA, 185 bp, Triticum aestivum
cgaaaagggcaacaagggtactacccaacttctctgcagcagccaggacaagggcaacaagggtactacccaacttctctgcagcacacaggacaaaggcaacaaccagtacaagggcaacaaccagaacaagggcaacaaccaggacaatggcaacaagggtactatccaacttctccacaacg

SEQ ID NO: 24 - DNA, 284 bp, Triticum aestivum
cggcgacaaggtcaacagtcagggcaagagcaacaagaacaacagataggacaagggcagcagccaggacaattgcaacaaccaacacaagggcaacaagggcagcaaccaggacaagggcaacaaggtcaacagccaggacaagggcaacaaggtcagcagccaggacaagggcagcaaccaggacaagggcagccagggtactacccaacttctttgcagcagtcaggacaagggcaacagccaggacaatggcaacaaccaggacaaggactaccaatcac

SEQ ID NO: 25 - DNA, 191 bp, Triticum aestivum
catccaggccaagcttctccccaacagtcaggacaaggacagcagccaggacaagaacagcaaccaggacaagggcaacaagatcagcagccaggacaaagacaacaaggatactacccaacttctccgcaacagccaggacaagggcaacaactgggacaagggcaaccagggtactacccaacttcacg

SEQ ID NO: 26 - protein, 307 aa, Triticum aestivum
RQQLGQGQQGYYPTSLQPGQKQQAGQGQQSGQGQQGYYPTSPQQSGQGQQPGQGQPGYYPTSPQQSGQWQQPGQGQQPGQGQQSGQWQLVYYPTSPQQQIVLQQRPPFLQQQQPSLPQQPPFSQQQQQLVLPQQQIPFVHPSILQQLNPCKVFLQQQCSPVAMPQSLARSQMLQQSSCHVMQQQCCQQLPQIPQQSRYEAIRAIIYSIILQEQQQVQGSIQTPQQQPQQLGQCVSQPQQQSQQQLGQQPQQQQLAQGTFLQPHQIAQLEVMTSIALRTLPTMCRVNVPLYRTTTSVPFGVGTGVGSY

SEQ ID NO: 27 - protein, 280 aa, Triticum aestivum
RNSGQQLGQGQPGYYPTSQQSEQRQQPGQGKQPGQGQQGYYPTSPQQSGQGQQLGQGQPGYYPTSPQQSGQGQQSGQGQQGYYPTSPQQSGQGQQPGQGQSGYFPTSRQQSGQGQQPGQGQQSGQGQQGQQPGQGQQAYYPTSSQQSRQRQQAGQWQRPGQGQPGYYPTSPQQPGQEQQSGQAQQSGQWQLVYYPTSPQQPGQLQQPAQGQQPAQGQQSAQEQQPGQAQQSGQWQLVYYPTSPQQPGQLQQPAQGQQGYYPTSPQQSGQGQQGYYPTSSR

SEQ ID NO: 28 - protein, 195 aa, Triticum aestivum
PTSPQQSGQGQQGYYPTSPQQSGQGQQGYYPTSPQQSGQGQQPGQGQQPRQGQQGYYPISPQQSGQGQQPGQGQQRYYPTSPQQSGQGQQPGHEQQPGQWLQPGQGQQGYYPTSSQQSGQGHQSGQGQQGYYPTSLWQPGQGQQPGQGQQGYASPYHVSAEYQAARLKVAKAQQLAAQLPAMCRLEGSDALSTRQ

SEQ ID NO: 29 - protein, 78 aa, Triticum aestivum
LSSGLSCPKGFPKLALESNSDEPDTIEYCNLGCRSSVCDYMVNAAADDEEMKLYVENCADACVSFCNGDAGLPSLDAY

SEQ ID NO: 30 - protein, 165 aa, Triticum aestivum
RNSQQPGQGQQGQQPGQGQQPGQGQPWYYPTSPQESGQGQQPGQWQQPGQGQPGYYLTSPLQLGQGQQGYYPTSLQQPGQGQQPGQWQQSGQGQHWYYPTSPQLSGQGQRPGQWLQPGQGQQGYYPTSPQQPGQGQQLGQWLQPGQGQQGYYPTSLQQTGQGQQS

SEQ ID NO: 31 - protein, 99 aa, Triticum aestivum
PGQWLQPGQGQQGYYPTSSQQSGQGHQSGQGQQGYYPTSLWQPGQGQQPGQGQQGYASPYHVSAEYQAARLKVAKAQQLAAQLPAMCRLEGSDALSTRQ

SEQ ID NO: 32 - protein, 86 aa, Triticum aestivum
QQQQQQQQPSSQVSYQQPQQQYPSGQGFFQPSQQNPQAQGFVQPQQLPQFEEIRNLALQTLPAMCNVYIPPYCSTTIAPFGIMSTN

SEQ ID NO: 33 - protein, 240 aa, Triticum aestivum
PQQPFPQTQQPQQPFPQQPQQPFPQTQQPQQPFPQQPQQPFPQTQQPQQPFPQLQQPQQPFPQPQQQLPQPQQPQQSFPQQQRPFIQPSLQQQLNPCKNILLQQCKPASLVSSLWSIIWPQSDCLVMRQQCCQQLAQIPQQLQCAAIHSVVHSIIMQQQQQQQQQQGMHIFLPLSQQQQVGQGSLVQGQGIIQPQQPAQLEAIRSLVLQTLPSMCNVYVPPECSIMRAPFASIVAGIGGQ

SEQ ID NO: 34 - protein, 270 aa, Triticum aestivum
RGQVQWPQQQPFRQPQQPFYQQPQHTFPQPQQTFPHQPQQQFPQPQQPQQQFPQPQQPQQPFPQPQQAQLPFPQQPQQPFPQPQQPQQPFPQSQQPQQPFPQPQQPQQSFPQQQQPLIQPYLQQQMETNPCKNYLLQQCNPVSLVSSLVSMETILPRSDCQVMETQQQCCQQLAQIPRQLQCAAIHSVVHSILMETQQEQQQGIQILRPLFQLVQGQGIIQPQQPAQYEVIRSLVLRTLPNMETCNVYVRPDCSTINAPFASIVAGISGQ

SEQ ID NO: 35 - protein, 111 aa, Triticum aestivum
PQQQFPQQQFHQQQLPQQQFPQQQFPQQQFPQQQQFPQQQQLTQQQFPRPQQSPEQQQFPQQQFPQQPPQQFPQQQFPIPYPPQQSQEPSPYQQYPQQQPSGSDVISISGL

SEQ ID NO: 36 - protein, 96 aa, Triticum aestivum
PLQRSGPELTIEMILQAAQPKLEPFPFEEHTDLPVQGLGGIGGGEVEDPTGGMGGEVQQDPTGGMGGEVQQDPTGGMGGEVEDPTGGMGGELPPTV

SEQ ID NO: 37 - protein, 75 aa, Triticum aestivum
RLLEPVSIGGSAAARAKFRAPQQQVPVEIMGMVLQTLPSMCSVNIPQYCTTTPCSTIAPAIYSIPMTATCAGGAC

SEQ ID NO: 38 - protein, 20 aa, Triticum aestivum
PQQQFPQPQQPQQPFPQQPQ

SEQ ID NO: 39 - protein, 34 aa, Triticum aestivum
PQQPQQQFPQSQQPQQPFPQPQQQFLQPQQPQQS

SEQ ID NO: 40 - protein, 31 aa, Triticum aestivum
PFPWQPQQPFPQTQQSFPLQPQQPFPQLPPS

SEQ ID NO: 41 - protein, 49 aa, Triticum aestivum
QQAQLPFPQQPQQPFPQPQQPQQPFPQSQQPQQPFPQPQQPQQSFPQLQ

SEQ ID NO: 42 - protein, 40 aa, Triticum aestivum
PFLQPQQPFPQQPQQPFPQTQQPQQPFPQQPQQPFPQTQQ

SEQ ID NO: 43 - protein, 75 aa, Triticum aestivum
PFLQPQQPFPQQPQQPFPQTQQPQQPFPQQPQQPFPQTQQPQQPFPQQPQQPFPQTQQPQQPFPQLQQPQQPFPQ

SEQ ID NO: 44 - protein, 79 aa, Triticum aestivum
QQFPQPQQPQQPFPQQPQQQFPQPQQPQQPFPQPQQPQLPFPQQPQQPFPQPQQPQQPFPQLQQPQQPLPQPQQPQQPF

SEQ ID NO: 45 - protein, 99 aa, Triticum aestivum
PSGQVQWLQQQLVPQLQQPLSQQPQQTFPQPQQTFPHQPQQQVPQPQQPQQPFLQPQQPFPQQPQQPFPQTQQPQQPFPRQLSAGRYHYQLVWCQRDPR

SEQ ID NO: 46 - protein, 38 aa, Triticum aestivum
PKDSAHRPLGRELRGGRPRHVGRTGLANGTGPWGRKRP

SEQ ID NO: 47 - protein, 97 aa, Triticum aestivum
PFLQPQQPFPQQPQQPFPQTQQPQQPFPQQPQQPFPQTQQPQQPFPQQPQQPFPQTQQPQQPFPQLQQPQQPFPQRIQPTARGEGSFEAADRGTSAG

SEQ ID NO: 48 - protein, 61 aa, Triticum aestivum
RKGQQGYYPTSLQQPGQGQQGYYPTSLQHTGQRQQPVQGQQPEQGQQPGQWQQGYYPTSPQ

SEQ ID NO: 49 - protein, 94 aa, Triticum aestivum
RRQGQQSGQEQQEQQIGQGQQPGQLQQPTQGQQGQQPGQGQQGQQPGQGQQGQQPGQGQQPGQGQPGYYPTSLQQSGQGQQPGQWQQPGQGLPI

SEQ ID NO: 50 - protein, 63 aa, Triticum aestivum
HPGQASPQQSGQGQQPGQEQQPGQGQQDQQPGQRQQGYYPTSPQQPGQGQQLGQGQPGYYPTS

SEQ ID NO: 51 - protein, 369 aa, Triticum aestivum
MKTFLIFALLAIVATSAIAQMENSHIPGLERPSQQQPLPPQQTLSHHQQQQPIQQQPQPFSQQQPCSQQQQQPLSQQQQPPFSQQQPPFSQQQQQPLSQQQQPPFSQQQPPFSQQQQPPFSQQQPPFSQQQQPVLPQQPSFSQQQLPPFSQQQSPFSQQQQIVLQQQPPFLQQQQPSLPQQPPFSQQQQQLVLPQQQIPFVHPSILQQLNPCKVFLQQQCSPVAMPQSLARSQMLQQSSCHVMQQQCCQQLPQIPQQSRYEAIRAIIYSIILQEQQQVQGSIQTPQQQPQQLGQCVSQPQQQSQQQLGQQPQQQQLAQGTFLQPHQIAQLEVMTSIALRTLPTMCRVNVPLYRTTTSVPFGVGTGVGSY

SEQ ID NO: 52 - protein, 795 aa, Triticum aestivum
MAKRLVLFAAVVVALVALTAAEGEASGQLQCEHELEACQQVVDQQLRDVSPGCRPITVSPGTRQYEQQPVVPSKAGSFYPSETTPSQQLQQMIFWGIPALLRRYYPSVTSSQQGSYYPGQASPQQSGQGQQPGQEQQPGQGQQDQQPGQRQQGYYPTSPQQPGQGQQLGQGQPGYYPTSQQPGQKQQAGQGQQSGQGQQGYYPTSPQQSGQGQQPGQGQPGYYPTSPQQSGQWQQPGQGQQPGQGQQSGQGQQGQQPGQGQRPGQGQQGYYPISPQQPGQGQQSGQGQPGYYPTSLRQPGQWQQPGQGQQPGQGQQGQQPGQGQQSGQGQQGYYPTSLQQPGQGQQLGQGQPGYYPTSQQSEQGQQPGQGKQPGQGQQGYYPTSPQQSGQGQQLGQGQPGYYPTSPQQSGQGQQSGQGQQGYYPTSPQQSGQGQQPGQGQSGYFPTSRQQSGQGQQPGQGQQSGQGQQGQQPGQGQQAYYPTSSQQSRQRQQAGQWQRPGQGQPGYYPTSPQQPGQEQQSGQAQQSGQWQLVYYPTSPQQPGQLQQPAQGQQPAQGQQSAQEQQPGQAQQSGQWQLVYYPTSPQQPGQLQQPAQGQQGYYPTSPQQSGQGQQGYYPTSPQQSGQGQQGYYPTSPQQSGQGQQPGQGQQPRQGQQGYYPISPQQSGQGQQPGQGQQGYYPTSPQQSGQGQQPGHEQQPGQWLQPGQGQQGYYPTSSQQSGQGHQSGQGQQGYYPTSLWQPGQGQQPGQGQQGYASPYHVSAEYQAARLKVAKAQQLAAQLPAMCRLEGSDALSTRQ

SEQ ID NO: 53 - protein, 137 aa, Triticum aestivum
MGSKGFKGVIVCLLILGLVLEQLQVEGKSCCRSTLGRNCYNLCRARGAQKLCAGVCRCKISSGLSCPKGFPKLALESNSDEPDTIEYCNLGCRSSVCDYMVNAAADDEEMKLYVENCADACVSFCNGDAGLPSLDAY

SEQ ID NO: 54 - protein, 839 aa, Triticum aestivum
MAKRLVLFVAVVVALVALTVAEGEASEQLQCERELQELQERELKACQQVMDQQLRDISPECHPVVVSPVAGQYEQQIVVPPKGGSFYPGATTPPQQLQQRIFWGIPALLKRYYPSVTSPQQVSYYPGQASPQRPGQGQQPGQGQQSGQGQQGYYPTSPQQPGQWQQPEQGQPGYYPTSPQQPGQLQQPAQGQQPGQGQQGRQPGQGQPGYYPTSSQLQPGQLQQPAQGQQGQQPGQGQQGQQPGQGQQPGQGQQGQQPGQGQQPGQGQQGQQLGQGQQGYYPTSLQQSGQGQPGYYPTSLQQLGQGQSGYYPTSPQQPGQGQQPGHLQQPAQGQQPEQGQQGQQPGQGQQGQQPGQGQQPGQGQPGYYPTSPQQSGQGQPGYYPTSSQRPTQSQQPGQGQQGQQVGQGQQAQQPGQGQQPGQGQPGYYPTSPLQSGQGQPGYYLTSPQQSGQGQQPGQLQQSAQGQKGQQPGQGQQPGQGQQGQQPGQGQQGQQPGQGQPGYYPTSPQQSGQGQQPGQWQQPGQGQPGYYPTSPLQPGQGQPVYDPTSPQQPGQGQQPGQLQQPAQGQQGQQLAQGQQGQQPAQVQQGQQPAQGQQGQQLGQGQQGQQPGQGQQPAQGQQGQQPGQGQQGQQPGQGQQPGQGQPWYYPTSPQESGLGQQPGQWQQPGQWQLPGQGQPGYYLTSPLQLGQGQQGYYPTSLQQPGQGQQPGQWQQSGQGQHGYYPTSPQLSGQGQRPGQWLQPGQGQQGYYPTSPQQSGQGQQLGQWLQPGQGQQGYYPTSLQQTGQGQQSGQGQQGYYSSYHVSVEHQAASLKVAKAQQLAAQLPAMCRLEGGDALSASQ

SEQ ID NO: 55 - protein, 713 aa, Triticum aestivum
MAKRLVLFGIVVIALVALTAAEGEASRQLQCERELQESSLEACRQVVDQQLAGRLPWSTGLQMRCCQQLRDVSAKCRRVAVSQVARQYEQTAVPPKGGSIYPGETTPLQQLQQGIFWGTSSQTVQGYYPSVTSPQQGSYYPGQASPQQPGQGQQQGKWQEPGQGQQGYYPTSQQQPGQGQQGHYPASQQQPGQGQQGHYPASLQQPGQGQQGHYPASLQQPGQGQQTEQPGQMQQPGQGQQIGQGQQPGQGQQIGQGQQIRQGQQPGQGQQGYYQTHPQQPGQGQQPGQGQQGYYPTSPQQPGQGQQGHYPGSLRQPGQGQPGQRQQPGQGQQTGQGQQPEQEQQPGQGQQGYYPTSPQQPGQGQQPGQGQQGYYPTSLQQPGQGQQPHYPASQQQPGQGQQGHYPTSLLQPGQGQQGHYPASSLQPGQGQQGHYPASLQQPGQGQQTEQPGQGQQPAQEQQSGQGQQGHYPTSLQQPGQGQPGQRQQPGQGQQIGQGQQPEQEQQPGQGQPGHYPASVQQPGQGQQTEQTGQGQQPGQGQQPEQEQQPGQGQQGYYITSLQQPGQGKQLGQWQQPGQGQEGYYPTSPQQPGQGQQGHCPTSRQQPGQAQQPGQGQQIGQAQKPGQGQQGYYPTSLQQPGQGQQSGQGNQPGQGHQPGQGQQSGQDQQGYDSPCHVSAEQKATSPKVAKAQQPVAQLPTMCQMEGGDTLSASQ

SEQ ID NO: 56 - protein, 307 aa, Triticum aestivum
MKTFLILALLAIVATTATIAVRVPVPQLQPQNPSQQQPQEQVPLVQQQQFPGQQQPFPPQQPYPQPQPFPSQQPYLQLQPFPQPQLPYPQPQLPYPQPQLPYPQPQPFRPQQPYPQSQPQYSQPQQPISQQQQQQQQQQQQKQQQQQQQQILQQILQQQLIPCRDVVLQQHSIAYGSSQVLQQSTYQLVQQLCCQQLWQIPEQSRCQAIHNVVHAIILHQQQQQQQQQQQQPLSQVSFQQPLQQYPLGQGSFQPSLQNPQAHGSVQPQQLPQFEEIRNLALQTLPAMCNVYIPPYCTIAPFGIFGTN

SEQ ID NO: 57 - protein, 311 aa, Triticum aestivum
MNIQVDPSGQVQWLQQQLVPQLQQPLSQQPQQTFPQPQQTFPHQPQQQVPQPQQPQQPFLQPQQPFPQQPQQPFPQTQQPQQPFPQQPQQPFPQTQQPQQPFPQQPQQPFPQTQQPQQPFPQLQQPQQPFPQPQQQLPQPQQPQQSFPQQQRSLIQPSLQQQLNPCKNILLQQCKPASLVSSLWSIIWPQSDCQVMRQQCCQQLAQIPQQLQCAAIHSVVHSIIMQQQQQQQQQQGMHIFLPLSQQQQVGQGSLVQGQAIIQPQQPAQLEAIRSLVLQTLPSMCNVYVPPECSIMRAPFASIVAGIGGQYR

SEQ ID NO: 58 - protein, 290 aa, Triticum aestivum
MKTLLILTIFAAALTIATANIQVDPSGQVQWPQQQPFPQPQPFSQQPQQAFLQPQHTFPLQPQQVFPQPQQPQQQFPQPQQPQQPFPQPQQPQLPFPQQPQQPFPQPQQPQQPFPQSQQPQQPFPQPQQQFPQPQQPQQSFPQQQPPLIQPYLQQQMNPCKNYLLQQCNPVSLVSSLVSMILPRNDCQVMQQQCCQQLAQIPRQLQCTAIHSVVHAIIMQQEQQGIQILRPLFQLVQGQGIIQPQQPAQYEVIRSLVLRTLPNMCNVYVRPDCSTINAPFASIVAGIGGQ

SEQ ID NO: 59 - protein, 439 aa, Triticum aestivum
MKTFIIFVLLAMAMNIASASRLLSPRGKELHTPQEQFPQQQQFPQPQQFPQQQIPQQHQIPQQPQQFPQQQQFLQQQQIPQQQIPQQHQIPQQPQQFPQQQQFPQQHQSPQQQFPQQQFPQQKLPQQEFPQQQISQQPQQLPQQQQIPQQPQQFLQQQQFPQQQPPQQHQFPQQQLPQQQQIPQQQQIPQQPQQIPQQQQIPQQPQQFPQQQFPQQQFPQQQFPQQEFPQQQQFPQQQIARQPQQLPQQQQIPQQPQQFPQQQQFPQQQSPQQQQFPQQQFPQQQQLPQKQFPQPQQIPQQQQIPQQPQQFPQQQFPQQQQFPQQQEFPQQQFPQQQFHQQQLPQQQFPQQQFPQQQFPQQQQFPQQQQLTQQQFPRPQQSPEQQQFPQQQFPQQPPQQFPQQQFPIPYPPQQSEEPSPYQQYPQQQPSGSDVISISGL

SEQ ID NO: 60 - protein, 533 aa, Hordeum vulgare
VNVKGNYVQVYVMLPLDAVSVNNRFEKGDELRAQLRKLVEAGVDGVMVDVWWGLVEGKGPKAYDWSAYKQLFELVQKAGLKLQAIMSFHQCGGNVGDAVNIPIPQWVRDVGTCDPDIFYTDGHGTRNIEYLTLGVDNQPLFHGRSAVQMYADYMTSFRENMKEFLDAGVIVDIEVGLGPAGEMRYPSYPQSHGWSFPGIGEFICYDKYLQADFKAAAAAVGHPEWEFPNDVGQYNDTPERTQFFRDNGTYLSEKGRFFLAWYSNNLIKHGDRILDEANKVFLGYKVQLAIKISGIHWWYKVPSHAAELTAGYYNLHDRDGYRTIARMLKRHRASINFTCAEMRDSEQSSQAMSAPEELVQQVLSAGWREGLNVACENALPRYDPTAYNTILRNARPHGINQSGPPEHKLFGFTYLRLSNQLVEGQNYANFKTFVDRMHANLPRDPYVDPMAPLPRSGPEISIEMILQAAQPKLQPFPFQEHTDLPVGPTGGMGGQAEGPTCGMGGQVKGPTGGMGGQAEDPTSGMGGELPATM

SEQ ID NO: 61 - protein, 168 aa, Triticum aestivum
MKTMFLLALLAFTATSAVAQLYTTCSQGYGQCQQQPQPQPQPQPQMNTCAAFLQQCSQTPHVQTQMWQASGCQLVRQQCCQPLAQISEQARCQAVCSVAQIIMRQQQGQSFGQPQQQVPVEIMRMVLQTLPLMCRVNIPQYCTTTPCSTITPAIYSIPMTATCAGGAC

SEQ ID NO: 62 - protein, 8 aa, Triticum aestivum
AQIPQQLQ

SEQ ID NO: 63 - protein, 12 aa, Triticum aestivum
PQQQRPFIQPSL

SEQ ID NO: 64 - protein, 17 aa, Triticum aestivum
LVQGQGIIQPQQPAQLE

SEQ ID NO: 65 - protein, 13 aa, Triticum aestivum
APFASIVAGIGGQ

SEQ ID NO: 66 - protein, 14 aa, Triticum aestivum
NIQVDPSGQVQALQ

SEQ ID NO: 67 - protein, 15 aa, Triticum aestivum
NIQVDPSGQVQWLQQ

SEQ ID NO: 68 - protein, 17 aa, Triticum aestivum
LVQGQGIIQPQQPAQLE

SEQ ID NO: 69 - protein, 16 aa, Triticum aestivum
LVPLSQQQQVGQGILV

SEQ ID NO: 70 - protein, 15 aa, Triticum aestivum
LPLYQQQQVGQGTLV

SEQ ID NO: 71 - protein, 16 aa, Triticum aestivum
FLPLSQQQQVGQGSLV

SEQ ID NO: 72 - protein, 12 aa, Triticum aestivum
LQLQPFPQPQLP

SEQ ID NO: 73 - protein, 24 aa, Triticum aestivum
FFQPSQQNPQAQGSFQPQQLPQFE

SEQ ID NO: 74 - protein, 22 aa, Triticum aestivum
FRPSQQNPQAQGSVQPQQLPQF

SEQ ID NO: 75 - protein, 20 aa, Triticum aestivum
RVPVPQLQPQNPSQQQPQKQ

SEQ ID NO: 76 - protein, 23 aa, Triticum aestivum
LQQHNIAHASSQVLQQSTYQLLQ

SEQ ID NO: 77 - protein, 22 aa, Triticum aestivum
LQQHNIAHGSSQVLQESTYQLV

SEQ ID NO: 78 - protein, 15 aa, Triticum aestivum
LQQHNIAHGSSQVLQ

SEQ ID NO: 79 - protein, 10 aa, Triticum aestivum
MVRVPVPQLQ

SEQ ID NO: 80 - protein, 21 aa, Triticum aestivum
LQQPNIAHASSQVSQQSYQLL

SEQ ID NO: 81 - protein, 11 aa, Triticum aestivum
FLQPHQIAQLE

SEQ ID NO: 82 - protein, 16 aa, Triticum aestivum
LAQGTFLQPHQIAQLE

SEQ ID NO: 83 - protein, 15 aa, Triticum aestivum
FSQQQQLFPQQPSFS

SEQ ID NO: 84 - protein, 15 aa, Triticum aestivum
LPQQQIPFVHPSILQ

SEQ ID NO: 85 - protein, 16 aa, Triticum aestivum
LPQQPPFSQQQQPILP

SEQ ID NO: 86 - protein, 15 aa, Triticum aestivum
LLQQQIPIVHPSILQ

SEQ ID NO: 87 - protein, 309 aa, Triticum aestivum
MNIQVDPSGQVQWLQQQLVPQLQQPLSQQPQQTFPQPQQTFPHQPQQQVPQPQQPQQPFLQPQQPFPQQPQQPFPQTQQPQQPFPQQPQQPFPQTQQPQQPFPQQPQQPFPQTQQPQQPFPQLQQPQQPFPQPQQQLPQPQQPQQSFPQQQRPFIQPSLQQQLNPCKNILLQQSKPASLVSSLWSIIWPQSDCQVMRQQCCQQLAQIPQQLQCAAIHSVVHSIIMQQQQQQQQQQGIDIFLPLSQHEQVGQGSLVQGQGIIQPQQPAQLEAIRSLVLQTLPSMCNVYVPPECSIMRAPFASIVAGIGGQ

SEQ ID NO: 88 - protein, 279 aa, Triticum aestivum
MNIQVDPSSQVQWPQQQPVPQPHQPFSQQPQQTFPQPQQTFPHQPQQQFPQPQQPQQQFLQPQQPFPQQPQQPYPQQPQQPFPQTQQPQQLFPQSQQPQQQFSQPQQQFPQPQQPQQSFPQQQPPFIQPSLQQQVNPCKNFLLQQCKPVSLVSSLWSMIWPQSDCQVMRQQSCQQLAQIPQQLQCAAIHTVIHSIIMQQEQQQGMHILLPLYQQQQVGQGTLVQGQGIIQPQQPAQLEAIRSLVLQTLPTMCNVYVPPECSIIKAPFSSVVAGIGGQYR

SEQ ID NO: 89 - protein, 19 aa, Triticum aestivum
MNIQVDPSGQVQWLQQQLV

SEQ ID NO: 90 - protein, 20 aa, Triticum aestivum
QQQLVPQLQQPLSQQPQQTF

SEQ ID NO: 91 - protein, 21 aa, Triticum aestivum
QQPQQTFPQPQQTFPHQPQQQ

SEQ ID NO: 92 - protein, 26 aa, Triticum aestivum
QPQQQVPQPQQPQQPFLQPQQPFPQQ

SEQ ID NO: 93 - protein, 18 aa, Triticum aestivum
PFPQQPQQPFPQTQQPQQ

SEQ ID NO: 94 - protein, 20 aa, Triticum aestivum
QQPQQPFPQQPQQPFPQTQQ

SEQ ID NO: 95 - protein, 20 aa, Triticum aestivum
PFPQTQQPQQPFPQLQQPQQ

SEQ ID NO: 96 - protein, 19 aa, Triticum aestivum
QQPQQPFPQPQQQLPQPQQ

SEQ ID NO: 97 - protein, 18 aa, Triticum aestivum
PQPQQPQQSFPQQQRPFI

SEQ ID NO: 98 - protein, 20 aa, Triticum aestivum
QRPFIQPSLQQQLNPCKNIL

SEQ ID NO: 99 - protein, 20 aa, Triticum aestivum
CKNILLQQSKPASLVSSLWS

SEQ ID NO: 100 - protein, 20 aa, Triticum aestivum
LVSSLWSIIWPQSDCQVMRQ

SEQ ID NO: 101 - protein, 21 aa, Triticum aestivum
QVMRQQCCQQLAQIPQQLQCA

SEQ ID NO: 102 - protein, 21 aa, Triticum aestivum
QLQCAAIHSVVHSIIMQQQQQ

SEQ ID NO: 103 - protein, 20 aa, Triticum aestivum
QQQQQQQQQQGIDIFLPLSQ

SEQ ID NO: 104 - protein, 20 aa, Triticum aestivum
LPLSQHEQVGQGSLVQGQGI

SEQ ID NO: 105 - protein, 21 aa, Triticum aestivum
QGQGIIQPQQPAQLEAIRSLV

SEQ ID NO: 106 - protein, 22 aa, Triticum aestivum
IRSLVLQTLPSMCNVYVPPECS

SEQ ID NO: 107 - protein, 21 aa, Triticum aestivum
PPECSIMRAPFASIVAGIGGQ

SEQ ID NO: 108 - protein, 13 aa, Triticum aestivum
LSQQQQVGQGSLV

SEQ ID NO: 109 - protein, 13 aa, Triticum aestivum
LYQQQQVGQGTLV

SEQ ID NO: 110 - protein, 15 aa, Triticum aestivum
LQQHSIAYGSSQVLQ